(19) United States
(12) Patent Application Publication
(10) Pub. No.: US 2021/0030468 A1
(43) Pub. Date: Feb. 4, 2021

(54) SYSTEMS AND METHODS FOR AUTOMATED GUIDANCE OF TREATMENT OF AN ORGAN

(71) Applicant: Navix International Limited, Road Town, Tortola (VG)

(72) Inventors: Zalman IBRAGIMOV, Rehovot (IL); Yitzhack SCHWARTZ, Haifa (IL); Yizhaq SHMAYAHU, Ramat-HaSharon (IL); Shlomo BEN-HAIM, Milan (IT)

(73) Assignee: Navix International Limited, Road Town, Tortola (VG)

(21) Appl. No.: 16/969,616

(22) PCT Filed: Feb. 14, 2019
(86) PCT No.: PCT/IB2019/051188

§ 371 Date: Aug. 13, 2020

Related U.S. Application Data
(60) Provisional application No. 62/630,332, filed on Feb. 14, 2018.

(51) Int. Cl. A61B 18/14; G16H 50/20; G16H 40/63; G16H 20/40

(52) U.S. Cl. CPC A61B 18/1492; G16H 50/20; A61B 2018/00577; G16H 20/40; G16H 40/63

(57) ABSTRACT

There is provided a computer implemented method of providing a client terminal with instructions for treatment of at least a portion of an organ of a patient, the method comprising: receiving electrical readings obtained by electrodes located within the portion of the organ, identifying by at least one classifier instructions for treatment of a region in the portion of the organ identified as an intervention target region, wherein the classifier identifies the instructions for treatment of the region based on electrical readings or a transformation thereof previously associated with treatment of intervention target regions in the portion of the organ of other patients, and marking on an image of the portion of the organ presented on a display, the instruction for treatment of the region identified by the classifier as an intervention target region.

### RELATED APPLICATION

This application claims the benefit of priority of U.S. Provisional Patent Application No. 62/630,332 filed on Feb. 14, 2018, the contents of which are incorporated herein by reference in their entirety.

### BACKGROUND

The present invention, in some embodiments thereof, relates to signal processing and, more specifically, but not exclusively, to systems and methods for machine learning methods for automated processing of electrical readings obtained within an organ.

Identification of the location and/or time and/or extent of administration of a medical intervention is performed, for example, during cardiac intervention (e.g., cardiac electrophysiologic treatment, cardiac vascular treatment, cardiac structural heart disease treatment (e.g., valvular), surgery, colonoscopy, biopsy, oncology surgery, orthopedic disk surgery, plastic surgery, and the like.

Users skilled in the medical arts may identify the tissue targets for the medical intervention and/or appropriate times for administration of the intervention based on training, special sense, luck, and the like. Experienced users are generally right most of the time. However, users at their beginning of training and/or lacking the skills and/or the tools have greater difficulty making an accurate and/or correct identification of the target tissue for administration of the medical intervention.

### SUMMARY

According to a first aspect, a computer implemented method of providing a client terminal with instructions for treatment of at least a portion of an organ of a patient, the method comprises: receiving electrical readings obtained by electrodes located within the portion of the organ, identifying by at least one classifier instructions for treatment of a region in the portion of the organ identified as an intervention target region, wherein the classifier identifies the instructions for treatment of the region based on electrical readings or a transformation thereof previously associated with treatment of intervention target regions in the portion of the organ of other patients, and marking on an image of the portion of the organ presented on a display, the instruction for treatment of the region identified by the classifier as an intervention target region.

According to a second aspect, a system for providing a client terminal with instructions for treatment of at least a portion of an organ of a patient, the system comprises: a non-transitory memory having stored thereon a code for execution by at least one hardware processor, the code comprising: code for receiving electrical readings obtained by electrodes located within the portion of the organ, code for identifying by at least one classifier instructions for treatment of a region in the portion of the organ identified as an intervention target region, wherein the classifier identifies the instructions for treatment of the region based on electrical readings or a transformation thereof previously associated with treatment of intervention target regions in the portion of the organ of other patients, and code for marking on an image of the portion of the organ presented on a display, the instructions for treatment of the location of a region identified by the classifier as an intervention target region.

According to a third aspect, a computer program product comprising a non-transitory computer readable storage medium storing program code thereon for implementation by a processor of a computing device for providing a client terminal with instructions for treatment of at least a portion of an organ of a patient, the program code comprises: instructions to receive electrical readings obtained by electrodes located within the portion of the organ, instructions to identify by at least one classifier instructions for treatment of a region in the portion of the organ identified as an intervention target region, wherein the classifier identifies the instructions for treatment of the region based on electrical readings or a transformation thereof previously associated with treatment of intervention target regions in the portion of the organ of other patients, and instructions to mark on an image of the portion of the organ presented on a display, the instructions for treatment of the location of a region identified by the classifier as an intervention target region.

According to a fourth aspect, A computer implemented method of training at least one classifier to identify instructions for treatment of at least one intervention target region of at least a portion of an organ of a target patient presented on a display of a client terminal, the method comprises: receiving for a plurality of sample individuals, electrical readings obtained by electrodes located within the portion of the organ of the respective sample individual, receiving for each of the plurality of sample individuals, an indication of treatment of a region in the portion of the organ identified as an intervention target region, wherein treatment of the intervention target region is associated with a subset of the electrical readings or a transformation thereof, training at least one classifier according to the subset of electrical readings or transformation thereof of the plurality of sample individuals and associated intervention target region, to identify, for a new target patient, instructions for treatment of an intervention target region based on electrical readings obtained for the new target patient or a transformation thereof, for presentation on an image presented on a display of the organ of the new target patient an indication of the identified intervention target region.

At least some implementations of the systems, apparatus, methods and/or code instructions (stored in a data storage device, executable by one or more hardware processors) described herein address the technical problem of guiding treatment of an intervention target region within an organ of a patient, where the user performing the treatment is not sufficiently experienced to accurately and/or safely perform the treatment. The particular solution to the technical problem is identifying by a trained classifier instructions for treatment according to electrical readings obtained by electrodes located within the organ.

At least some implementations of the systems, apparatus, methods and/or code instructions (stored in a data storage device, executable by one or more hardware processors) described herein are directed to an improvement in computer-related technology, by allowing computers to automatically identify instructions for treatment of region(s) of an organ. Such instructions for treatment previously could only be provided in real-time by particular humans performing the interventional procedure, for example, physicians that spent many years in performing the treatments, for example, learning to identify the interventional target regions, and treat them. Such humans manually identify and treat the intervention target region based on previous training, gut instinct, an educated guess, and/or based on consultation with other colleagues. However, it is noted that the systems, apparatus, methods and/or code instructions described herein are not a computer-implemented version of a mental process, and are not intended to replicate or model human capability, but provide an improvement in the ability to analyze a large number of electrical signals and automatically identify instructions for treatment of intervention target regions that would otherwise could not be performed by the particular user. Humans are unable to synthesize and analyze a large number of such electrical signals, instead relying on a small number of sample points, which may generate inaccurate and/or incomplete results for example, when the human has not seen a similar case before. The systems, apparatus, methods and/or code instructions described herein, which operate differently than a human operates in identifying instructions for treatment of the intervention target region, by consideration of a large number of electrical readings and based on a large number of previously defined associations (which may be larger than a human may possible experience in a lifetime), may provide more accurate delineations of instructions for treatment of the interventional target region(s) in comparison to the human ability, and/or may identify instructions for treatment of intervention target region(s) which would not otherwise be identified by the human user.

At least some implementations of the systems, apparatus, methods and/or code instructions described herein generate a new user experience, one that is different than mentally trying to identify instructions for treatment of the intervention target region based on a small number of measurements according to common practice. For example, the user manipulates the catheter to obtain a large number of electrical readings within the heart. A 3D image of the portion of the organ may be automatically presented, on which is automatically marked the instructions for treatment of the intervention target region. The user may be presented with recommendations for treatment, and/or guided in treatment, according to the identified instructions for treatment of the intervention target region.

At least some implementations of the systems, apparatus, methods and/or code instructions (stored in a data storage device, executable by one or more hardware processors) described herein may shorten the medical intervention, and this way reduce the number of complications, and ease the recovery of the patient from the operation. For example, in an ablation operation aimed at generating electrical isolation between the pulmonary veins and the left atrium, a physician may achieve the isolation with a smaller number of better positioned ablations, than would be required in absence of the system's guidance.

In a further implementation form of the first, second, third, and fourth aspects, the instructions include one or more members selected from the group consisting of: a text message presented on the display indicating how to treat the intervention target region, an animation simulating treatment of the intervention target region, a video captured of another user previously performing a treatment, and an audio recording of instructions how to treat the intervention target region.

In a further implementation form of the first, second, third, and fourth aspects, the instructions provide directions for treatment according to a hardware type of a treatment device for performing the treatment.

In a further implementation form of the first, second, third, and fourth aspects, the instructions for treatment are identified according to a hardware type of a treatment device that is used to apply a treatment to the patient selected from the group consisting of: probe pressure, heating, cooling, cardiac pacing, defibrillation, radiofrequency energy application, radiofrequency ablation, cryo application, cryo ablation, other energy delivery, and combinations of the aforementioned.

In a further implementation form of the first, second, and third aspects, the method further comprises and/or the system further comprises code instructions for and/or the computer program product further comprises additional instructions for reconstructing the image of the portion of the organ based on the electrical readings or the transformation thereof.

In a further implementation form of the first, second, third, and fourth aspects, the electrical readings are of a first type, the image is computed based on a second type of electrical reading, and the electrical readings of the first type are associated with locations at which the electrical readings were obtained by the electrodes, the locations associated with the electrical readings of the first type are mapped to corresponding locations of the image.

In a further implementation form of the first, second, third, and fourth aspects, the first type is an electrogram and the second type is an impedance reading.

In a further implementation form of the first, second, third, and fourth aspects, the electrical readings comprise impedance electrical readings indicative of impedance of tissue touching at least one of the electrodes when the impedance electrical readings are read.

In a further implementation form of the first, second, and third, aspects, the classifier identifies instructions for treatment of the region based on impedance electrical readings previously associated with treatment of intervention target regions in the portion of the organ of other patients.

In a further implementation form of the first, second, third, and fourth aspects, the electrical readings comprise position electrical readings indicative of anatomical position of at least one of the electrodes when the position electrical readings are read, wherein each anatomical position is associated with an anatomical structure.

In a further implementation form of the first, second, and third aspects, the classifier identifies instructions for treatment of the region based on position electrical readings previously associated with treatment of intervention target regions in the portion of the organ of other patients, wherein each position electrical reading is in reference to a 3D coordinate system within which the organ is located.

In a further implementation form of the first, second, and third aspects, the classifier identifies instructions for treatment of the region based on anatomical positions previously associated with treatment of intervention target regions in the portion of the organ of other patients.

In a further implementation form of the first, second, and third aspects, the classifier identifies instructions for treatment of the region as an intervention target region based on combinations of anatomical positions and impedance electrical readings, said combinations being previously associated with treatment of intervention target regions in the portion of the organ of other patients.

In a further implementation form of the first, second, third, and fourth aspects, the electrical readings comprise cardiac-phased electrical readings, each being an electrical reading associated with an indication as to where on a cardiac cycle the electrical reading was obtained.

In a further implementation form of the first, second, and third aspects, the classifier identifies instructions for treatment of the region based on cardiac-phased electrical readings, previously associated with treatment of intervention target regions in the portion of the organ of other patients.

In a further implementation form of the first, second, third, and fourth aspects, the electrical readings comprise respiratory-phased electrical readings, each being an electrical reading associated with an indication as to where on a respiratory cycle the electrical reading was obtained.

In a further implementation form of the first, second, and third aspects, the classifier identifies instructions for treatment of the region as an intervention target region based on respiratory-phased electrical readings, previously associated with treatment of intervention target regions in the portion of the organ of other patients.

In a further implementation form of the first, second, third, and fourth aspects, each of at least one of the electrical readings is both respiratory-phased and cardiac-phased.

In a further implementation form of the first, second, third, and fourth aspects, at least one electrical reading is an electrogrammed electrical reading, associated with an electrogram obtained by at least one of the electrodes when the at least one electrical reading was obtained.

In a further implementation form of the first, second, and third aspects, the classifier identifies instructions for treatment of the region based on electrogrammed electrical readings, previously associated with treatment of intervention target regions in the portion of the organ of other patients.

In a further implementation form of the first, second, and third aspects, each of the electrical readings is associated with a time stamp indicative of the time at which the respective electrical reading was obtained by the electrodes, wherein the electrical readings having time stamps within defined time windows indicative of an approximate simultaneous time of acquisition are clustered to time window clusters, wherein the classifier identifies the instructions for treatment of intervention target region according to time window clusters of electrical readings or a transformation thereof previously associated with treatment of intervention target regions in the portion of the organ of other patients.

In a further implementation form of the first, second, and third aspects, the method further comprises and/or the system further comprises code instructions for and/or the computer program product further comprises additional instructions for receiving a profile of the patient, wherein the classifier identifies instructions for treatment of the region based on resemblance between the profile of the patient and profiles of the other patients.

In a further implementation form of the first, second, third, and fourth aspects, the profile includes one or more data elements selected from the group comprising: clinical data, demographic data, gender, age, body mass index (BMI), and combinations of the aforementioned.

In a further implementation form of the first, second, and third aspects, the classifier identifies instructions for treatment of the region as an intervention target region based on previously observed associations between scores indicative of success of the treatment of intervention target regions in the portion of the organ of other patients, and wherein the identifying instructions for treatment by the classifier is performed according to maximization of a predicted likelihood of success of a treatment performed according to the identified intervention target region.

In a further implementation form of the first, second, third, and fourth aspects, the instructions for treatment of the target intervention region denotes instructions for ablation treatment of an ablation region, and wherein the ablation region is marked on the image of the portion of the organ.

In a further implementation form of the first, second, third, and fourth aspects, the ablation region is an ablation line.

In a further implementation form of the first, second, third, and fourth aspects, the ablation region denotes an ablation region for pulmonary vein isolation (PVI) ablation.

In a further implementation form of the first, second, third, and fourth aspects, identifying comprises identifying by the at least one classifier instructions for treatment of a plurality of regions identified as intervention target regions, wherein the plurality of regions define an ablation region.

In a further implementation form of the first, second, and third aspects, the method further comprises and/or the system further comprises code instructions for and/or the computer program product further comprises additional instructions for identifying by at least one classifier instructions for avoidance of treatment of a second region in the portion of the organ identified as a non-intervention region indicative of a region in which intervention is prohibited, wherein the classifier is based on observed associations between previously analyzed electrical readings and regions in the portion of the organ previously identified as non-intervention regions where treatment was avoided.

In a further implementation form of the first, second, third, and fourth aspects, identifying comprises identifying by the at least one classifier instructions for treatment of a plurality of regions, each region identified as a target region for a certain type of intervention selected from a plurality of types of interventions, wherein each region of each type of intervention target region of the plurality of types of intervention target regions is identified based on a certain subset of the received electrical reading.

In a further implementation form of the first, second, third, and fourth aspects, the plurality of regions are marked on the image of the portion of the organ with distinct identifiers according to each of the plurality of types of intervention target regions.

In a further implementation form of the first, second, third, and fourth aspects, one of the plurality of regions is marked on the image portion of the organ according to a selection of one of the plurality of types of intervention target regions.

In a further implementation form of the first, second, third, and fourth aspects, each of the plurality of types of intervention target regions denotes a respective ablation line identified based on different criteria.

In a further implementation form of the first, second, third, and fourth aspects, each of the plurality of types of intervention target regions denoting a respective ablation line for pulmonary vein ablation is based on one set of the following criteria: (i) electrical readings from the pulmonary artery and left atrium being equal according to an equality requirement, (ii) maximum simultaneous values of the electrical readings from the pulmonary artery and left atrium according to a maximal requirement, (iii) presence of the left atrium electrical reading and absence of the pulmonary artery electrical reading within a presence-absence requirement.

In a further implementation form of the first, second, and third aspects, the method further comprises and/or the system further comprises code instructions for and/or the computer program product further comprises additional instructions for receiving additional electrical readings obtained by the electrodes after at least one tissue region of the organ is treated, re-identifying by the at least one classifier updated instructions for treatment of a region in the portion of the organ identified as an updated intervention target region, wherein the instructions for treatment of region are updated by the at least one classifier based on observed associations between previously analyzed electrical readings and treatment of regions in the portion of the organ previously identified as updated intervention target regions, and marking on the image of the portion of the organ, instructions for treatment of an updated region identified by the at least one classifier as an updated target region.

In a further implementation form of the first, second, and third aspects, the method further comprises and/or the system further comprises code instructions for and/or the computer program product further comprises additional instructions for receiving an indication of a treatment region in which an intervention treatment was performed within the portion of the organ, computing an adjustment to the instructions for treatment of the intervention target region identified by the at least one classifier according to the treatment region, and marking the adjusted instructions on the image.

In a further implementation form of the first, second, and third aspects, the method further comprises and/or the system further comprises code instructions for and/or the computer program product further comprises additional instructions for dynamically computing an indication of a recommendation for proceeding in the treatment to treat the intervention target region according to the updated intervention target region, for presentation in association with the image.

In a further implementation form of the first, second, third, and fourth aspects, the additional electrical readings are obtained after at least a portion of an interventional procedure is performed according to the location of the region identified by the classifier as the intervention target region.

In a further implementation form of the first, second, third, and fourth aspects, the instructions include treatment of the intervention target region corresponding to a fossa Ovalis.

In a further implementation form of the first, second, third, and fourth aspects, the electrical readings are selected from the group consisting of readings of: voltage, impedance, endocardial electrical activity, electrical activity, dielectric property, S-parameter and combinations of the aforementioned.

In a further implementation form of the first, second, third, and fourth aspects, the electrical readings are generated by one or more of the following: patch electrodes located externally to the body of the target individual that apply a plurality of alternating currents each at a respective frequency, electrodes on an intra-body catheter located in proximity to the electrodes that obtain the electrical readings, and electrodes on an intra-body catheter located in a predefined anatomical region.

In a further implementation form of the first, second, and third aspects, the method further comprises and/or the system further comprises code instructions for and/or the computer program product further comprises additional instructions for receiving at least one tissue property obtained by at least one sensor located within the portion of the organ,

wherein identifying instructions for treatment of the region by the at least one classifier is further based on observed associations between the at least one tissue property and treatment of regions in the portion of the organ of other patients identified as intervention target regions.

In a further implementation form of the first, second, third, and fourth aspects, the at least one tissue property is selected from the group consisting of: molecular structure, IR reflectance, NIR reflectance, Ho Yag reflectance, pH, Ion concentration, Reactance, tissue thickness, scars and combinations of the aforementioned.

In a further implementation form of the first, second, and third aspects, the method further comprises and/or the system further comprises code instructions for and/or the computer program product further comprises additional instructions for receiving manually entered instructions for treatment of a designated intervention target region, correlating the instructions for treatment of the intervention target region identified by the at least one classifier with the manually entered instructions for treatment of the intervention target region according to a correlation requirement, computing an adjustment to the manually entered instructions for treatment of the intervention target region that satisfies the correlation requirement, and providing an indication of the adjustment of the instructions for treatment.

In a further implementation form of the first, second, and third aspects, the instructions for treatment include: predicting, by the at least one classifier, power of ablation and time of application of ablation energy for performing an intervention procedure according to the identified intervention target region, said predicting being based on observed associations between intervention target regions and previously analyzed powers of ablation and times of application of ablation energy.

In a further implementation form of the first, second, and third aspects, the instructions for treatment include: estimating by the at least one classifier a number of catheter manipulations for performing the intervention procedure according to the geometry of the region identified as the intervention target region based on associations between previously analyzed number of catheter manipulation for performing the intervention procedure and regions previously identified as intervention target regions.

In a further implementation form of the fourth aspect, the method further comprises: receiving, for a new sample individual, new electrical readings obtained by electrodes located within the portion of the organ of the new sample individual, receiving for the new sample individual, an indication of treatment of a region in the portion of the organ identified as an intervention target region, wherein the treatment of the intervention target region is associated with a subset of the new electrical readings or a transformation thereof, and updating the at least one classifier according to the indication of the treatment of the intervention target region based on new electrical readings or transformation thereof of the new sample individual.

In a further implementation form of the fourth aspect, the method further comprises: receiving an indication of a certain type of anatomical variation of a plurality of anatomical variations for each of the sample individuals, clustering the sample individuals according to each of the plurality of anatomical variations, wherein sample individual members of each cluster have a similar type of anatomical variation, wherein the at least one classifier is trained according to the electrical readings or transformation thereof of sample individual members of each cluster and associated treatment of intervention target region, for identifying at instructions for treatment of least one intervention target region for the new target patient associated with an indication of one of the plurality of anatomical variations.

In a further implementation form of the fourth aspect, the certain type of anatomical variation of each of the sample individuals is retrieved from an electronic medical record of the sample individual storing pre-identified anatomical variations.

In a further implementation form of the fourth aspect, the method further comprises: obtaining medical data for the plurality of sample individuals obtained at least a time interval after completion of the intervention procedure during which the electrical readings were obtained, updating the at least one classifier according to the obtained medical data associated with each of the plurality of sample individuals, wherein the at least one classifier identifies for the new target patient the instructions for treatment of the intervention target region based on electrical readings or a transformation thereof according to a target result of the medical data.

In a further implementation form of the fourth aspect, the method further comprises: obtaining medical data for the plurality of sample individuals obtained at least a time interval after completion of the intervention procedure during which the electrical readings were obtained, updating the at least one classifier by removing data of sample individuals associated with an indication of an unsuccessful procedure outcome.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1A is a flowchart of a method of providing a client terminal with instructions for treatment of at least a portion of an organ of a patient, in accordance with some embodiments of the present invention;

FIG. 1B is a flowchart of a method of training one or more classifier(s) for identifying instructions for treatment of intervention target region(s), in accordance with some embodiments of the present invention;

FIG. 2 is a block diagram of components of a system for identifying instructions for treatment of one or more intervention target regions by a classifier, in accordance with some embodiments of the present invention;

FIG. 3 is a schematic of a 3D image of a left atrium and pulmonary veins and regions from which electrical readings for identification of instructions for treatment an intervention target region were obtained by respective electrodes, in accordance with some embodiments of the present invention;

FIG. 4 is a schematic depicting an exemplary process of generating a classifier(s) for identifying instructions for treatment of region(s) in a portion of an organ identified as intervention target region(s), in accordance with some embodiments of the present invention; and

FIG. 5 is a flowchart depicting an exemplary process of identifying instructions for treatment of a region in a portion of an organ as an ablation line target region, in accordance with some embodiments of the present invention.

### DETAILED DESCRIPTION

The present invention, in some embodiments thereof, relates to signal processing and, more specifically, but not exclusively, to systems and methods for machine learning methods for automated processing of electrical readings obtained within an organ.

An aspect of some embodiments of the present invention relates to systems, methods, an apparatus, and/or code instructions (e.g., stored in a data storage device executable by one or more hardware processors) for providing a client terminal with instructions for treatment of at least a portion of an organ (e.g., heart) of a target patient. In some embodiments, the instructions for treatment are used to guide a physician to carry out a medical intervention (e.g., an operation, ablation procedure, etc.) following a similar intervention carried out by one or more experienced physicians. Accordingly, in some embodiments, an image of the organ marked with instructions for treatment is generated based on imaging of a current patient, and instructions based on interventions made with other patients by the experienced physician(s). The instructions used for guiding the current physician, may be identified by a classifier(s) which has been trained according to data gathered during similar medical interventions performed by the more experienced physician(s) on multiple other patients. Effectively, the less experienced physician is guided by the instructions to emulate behavior of the experienced physician(s), without real-time communication between the less experienced physician and the more experienced physician(s). The less experience physician may be guided by the instructions in real-time according to progress of the medical intervention. The classifier(s) may suggest an initial treatment plan, and may also suggest changes to the medical intervention when the medical intervention being performed by the less experienced physician deviates from the initial treatment plan, or when additional data is gathered during the operation, and the classifier finds that considering this new data, a change to the plan is appropriate. The classifier(s) may guide physicians in performing medical procedures that are seldom performed, where the number of specialists performing such medical procedures is small, and/or in geographic locations where access to such specialists is not available, and therefore the patient is being treated by the less experienced physician.

Exemplary instructions include one or more of the following: a marking of the intervention target region on an image of the organ, a window on a display presenting settings of the treatment device for performing the treatment (e.g., settings of power, time, pattern of energy delivery), a text message presented on the display with advice for example “apply energy to the intervention target region in a posterior approach”, an animation depicting a simulation of the treatment, a video recorded of a previous expert physician performing a similar treatment on another patient, and an audio message (synthesized voice and/or recording of an expert physician) played over speakers.

In some embodiments, electrical readings(s) are obtained by electrodes located within the portion of the organ, for example, on a distal end portion of a catheter. One or more classifiers identify one or more regions in the portion of the organ as an intervention target region(s) based on the electrical reading(s) and/or transformation of the electrical reading(s) previously associated with intervention target region(s) in the portion of the organ of other sample patients. The location of the region identified by the classifier(s) as the intervention target region(s) is marked on a 3D image of the portion of the organ, to guide the physician to intervene at the identified intervention target region(s).

Optionally, the interventional target regions(s) include an ablation region, for example, an ablation line. The ablation region may be ablated, for example, by application of radiofrequency (RF) energy, application of cryoenergy and/or other ablation energies.

An aspect of some embodiments of the present invention relates to systems, methods, an apparatus, and/or code instructions (e.g., stored in a data storage device executable by one or more hardware processors) for training one or more classifier(s) to identify instructions for treatment of at least a portion of an organ of a target patient. The classifier(s) is trained based on electrical readings(s) obtained for each of multiple sample patients, by electrodes located within the portion of the organ of the respective sample patient. An indication of a treatment scheme is received for each of the sample individuals, for example, by an operator (e.g., the expert physician) manually marking the treatment scheme on a graphical user interface (GUI).

Optionally, the instructions for treatment include identification of one or more intervention treatment regions of the organ to which the treatment is applied. The intervention treatment regions may be provided, for example, by the operating manually marking the intervention treatment regions on the GUI. The intervention target region is associated with a subset of the electrical readings, or a subset of a transformation of the electrical readings, for example, a region marked on the GUI that includes a subset of the electrical readings presented on the GUI as dots. The subset of electrical readings represents the measurements on which the expert physician basis the manual identification of the intervention target region. One or more classifiers are trained according to the subset of electrical readings (or the full set of electrical readings) and/or the subset of transformation(s) of the electrical readings (or the full set of transformations) and the associated marked intervention target region.

Some implementations of the systems, apparatus, methods and/or code instructions (stored in a data storage device, executable by one or more hardware processors) described herein address the technical problem of guiding treatment of an intervention target region within an organ of a patient, where the user performing the treatment is not sufficiently experienced to accurately and/or safely perform the treatment. The particular solution to the technical problem is identifying by a trained classifier instructions for treatment according to electrical readings obtained by electrodes located within the organ.

Some implementations of the systems, apparatus, methods and/or code instructions (stored in a data storage device, executable by one or more hardware processors) described herein are directed to an improvement in computer-related technology, by allowing computers to automatically identify instructions for treatment of region(s) of an organ. Such instructions for treatment previously could only be provided in real-time by particular humans performing the interventional procedure, for example, physicians that spent many years in performing the treatments, for example, learning to identify the interventional target regions, and treat them. Such humans manually identify and treat the intervention target region based on previous training, gut instinct, an educated guess, and/or based on consultation with other colleagues. However, it is noted that the systems, apparatus, methods and/or code instructions described herein are not a computer-implemented version of a mental process, and are not intended to replicate or model human capability, but provide an improvement in the ability to analyze a large number of electrical signals and automatically identify instructions for treatment of intervention target regions that would otherwise could not be performed by the particular user. Humans are unable to synthesize and analyze a large number of such electrical signals, instead relying on a small number of sample points, which may generate inaccurate and/or incomplete results for example, when the human has not seen a similar case before. The systems, apparatus, methods and/or code instructions described herein, which operate differently than a human operates in identifying instructions for treatment of the intervention target region, by consideration of a large number of electrical readings and based on a large number of previously defined associations (which may be larger than a human may possible experience in a lifetime), may provide more accurate delineations of instructions for treatment of the interventional target region(s) in comparison to the human ability, and/or may identify instructions for treatment of intervention target region(s) which would not otherwise be identified by the human user.

Some implementations of the systems, apparatus, methods and/or code instructions described herein generate a new user experience, one that is different than mentally trying to identify instructions for treatment of the intervention target region based on a small number of measurements according to common practice. For example, the user manipulates the catheter to obtain a large number of electrical readings within the heart. A 3D image of the portion of the organ may be automatically presented, on which is automatically marked the instructions for treatment of the intervention target region. The user may be presented with recommendations for treatment, and/or guided in treatment, according to the identified instructions for treatment of the intervention target region.

Some implementations of the systems, apparatus, methods and/or code instructions (stored in a data storage device, executable by one or more hardware processors) described herein may shorten the medical intervention, and this way reduce the number of complications, and ease the recovery of the patient from the operation. For example, in an ablation operation aimed at generating electrical isolation between the pulmonary veins and the left atrium, a physician may achieve the isolation with a smaller number of better positioned ablations, than would be required in absence of the system's guidance.

Some implementations of the systems, apparatus, methods and/or code instructions (stored in a data storage device, executable by one or more hardware processors) described herein improve an underlying technical process within the technical field of signal processing and/or within the technical field of machine learning.

Some implementations of the systems, apparatus, methods and/or code instructions (stored in a data storage device, executable by one or more hardware processors) described herein improve an underlying technical process within the technical field of planning medical treatment plans and executing them.

Some implementations of the systems, apparatus, methods and/or code instructions (stored in a data storage device, executable by one or more hardware processors) described herein automatically generate new data in the form of the instructions for treatment of the identified intervention target region, which as discussed above, has not been previously performed by a computer but has been identified manually by a user.

Some implementations of the systems, apparatus, methods and/or code instructions (stored in a data storage device, executable by one or more hardware processors) described herein are tied to physical real-life components, for example, electrodes located on a catheter that perform the electrical readings are physical real-life components, a display is a physical real-life component, and the hardware processor(s) that executes code instructions, as well as the memory storage device storing the code instructions, are all physical real-life components.

Some implementations of the systems, apparatus, methods and/or code instructions (stored in a data storage device, executable by one or more hardware processors) described herein provide a unique, particular, and advanced technique of identifying instructions for treatment of a region of an organ.

Accordingly, some implementations of the systems and/or methods described herein are inextricably tied to computer technology.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

As used herein, the term electrical readings may sometimes be interchanged with the phrase transformation(s) of the electrical readings. For example, the classifier may receive as input the electrical readings and/or transformation of the electrical readings. The transformation of the electrical readings may include a physical quantity calculated based on the electrical readings. For example, an electrical reading may be transformed to a location within a space at which the electrode that obtained the electrical reading is located. The classifier may receive as input the electrical reading and/or the location to which this electrical reading is transformed. In some embodiments, electrical readings may be transformed into locations as described in PCT patent application PCT/IB2017/056616 or PCT/TB 2018/050192.

As used herein, the term classifier may refer to one or multiple classifiers and/or artificial intelligence code. For example, multiple classifiers may be trained, which may process data in parallel and/or as a pipeline. For example, output of one type of classifier (e.g., from intermediate layers of a neural network) is fed as input into another type of classifier. Exemplary classifiers include: one or more neural networks of various architectures (e.g., artificial, deep, convolutional, fully connected), support vector machine (SVM), logistic regression, k-nearest neighbor, and decision trees.

As used herein, the term 3D image refers to an exemplary embodiment, and is not necessarily meant to limit the image to 3D. It is noted that other images may be substituted for the term 3D image, for example, 2D images and 4D images (where the fourth dimension may be time).

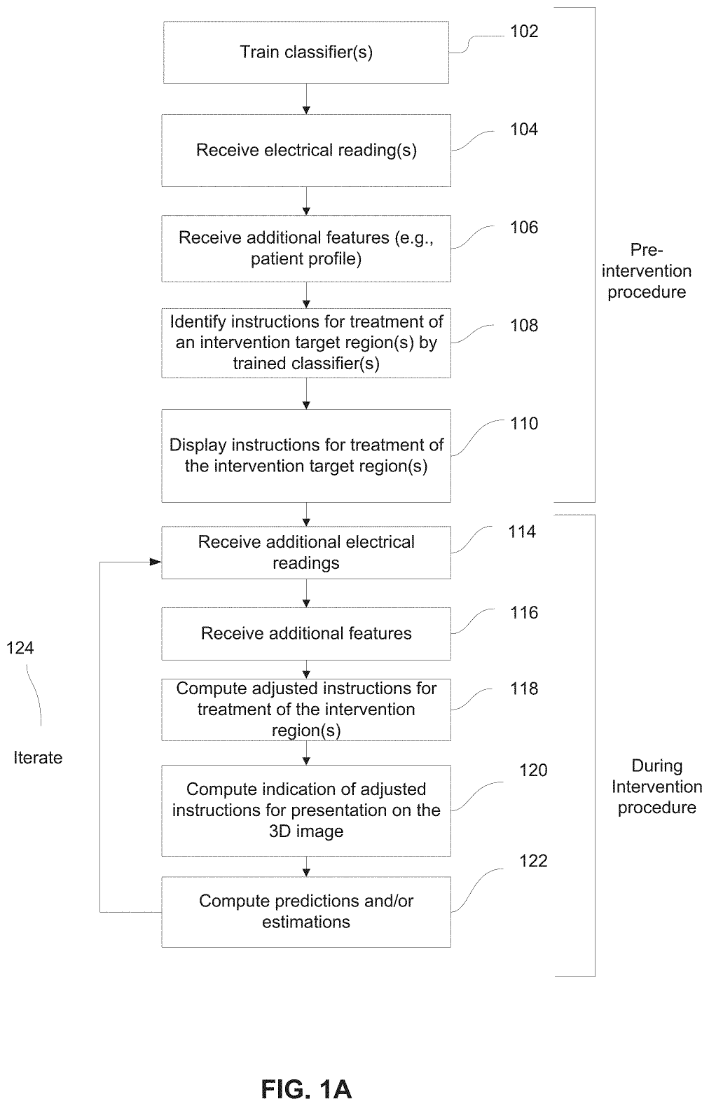

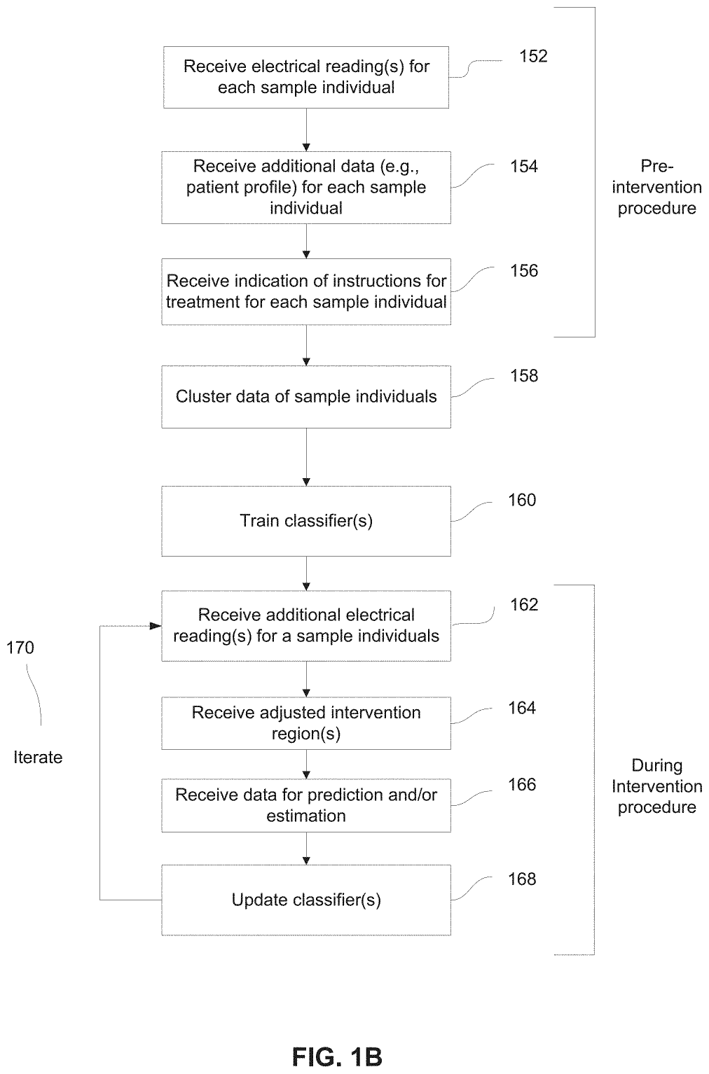

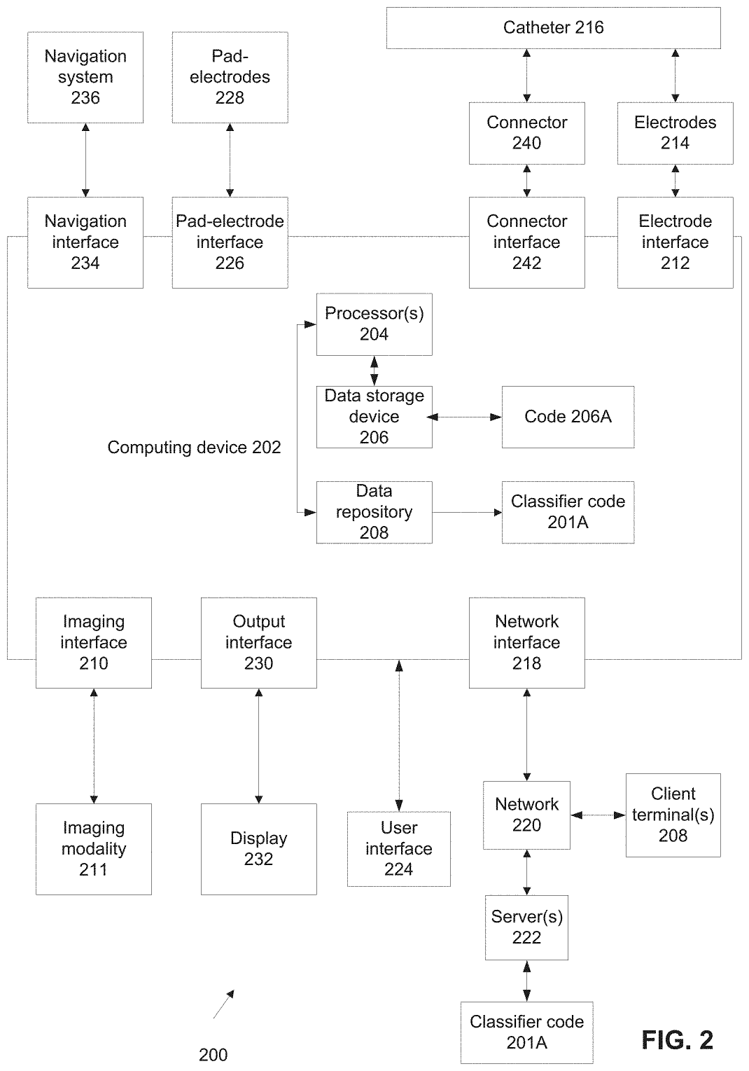

Reference is now made to FIG. 1A, which is a flowchart of a method of providing a client terminal with instructions for treatment of at least a portion of an organ of a patient, in accordance with some embodiments of the present invention. Reference is also made to FIG. 2, which is a block diagram of components of a system 200 for identifying instructions for treatment of one or more intervention target regions by a classifier 201A, in accordance with some embodiments of the present invention. Reference is also made to FIG. 1B, which is a flowchart of a method of training one or more classifier(s) for identifying instructions for treatment of at least one intervention target region, in accordance with some embodiments of the present invention. System 200 may implement the acts of the method described with reference to FIGS. 1A-B, optionally by a hardware processor(s) 204 of a computing device 202 executing code instructions stored in a data storage device 206.

System 200 may include code instructions for training classifier(s) 201A. The training code instructions may be stored in a storage device, for example, storage device 206 and/or 208. Alternatively, classifier 201A is trained by another computing device (e.g., server 222) and transmitted to computing device 202 and/or remotely accessed by computing device 202 (e.g., via a network 220, and/or via a software interface, for example, application programming interface (API), and/or software development kit (SDK)).

Computing device 202 may be implemented as, for example, a client terminal, a server, a computing cloud, a virtual machine, a radiology workstation, a workstation installed within a catheterization laboratory, a mobile device, a desktop computer, a thin client, a Smartphone, a Tablet computer, a laptop computer, a wearable computer, glasses computer, and a watch computer.

Multiple architectures of system 200 based on computing device 202 may be implemented. For example, computing device 202 may be implemented as an existing device (e.g., client terminal) having software (e.g., code 206A) that performs one or more of the acts described with reference to FIGS. 1A-B, for example, code 206A is installed on a computer conventionally existing in a catheterization/interventional lab. In another implementation, computing device 202 may be implemented as a dedicated device, having software (e.g., code 206A) installed thereon. In another exemplary implementation, computing device 202 storing code 206A may be implemented as one or more servers (e.g., network server, web server, a computing cloud, a virtual server, a radiology server, an interventional laboratory server) that provides services (e.g., one or more of the acts described with reference to FIGS. 1A-B) to one or more client terminals 208 over network 220. Client terminal 208 may be, in some embodiments, a terminal located remotely from computing device 202, for example, an interventional/catheterization laboratory client having access to the server.

Hardware processor(s) 204 may be implemented for performing the acts of the method described with reference to FIGS. 1A-B. In some embodiments, hardware processor(s) 204 may be implemented as a central processing unit(s) (CPU), a graphics processing unit(s) (GPU), field programmable gate array(s) (FPGA), digital signal processor(s) (DSP), and/or application specific integrated circuit(s) (ASIC). Processor(s) 204 may include one or more processors (homogenous or heterogeneous), which may be arranged for parallel processing, as clusters and/or as one or more multi core processors. Data storage device 206 stores code instructions executable by processor(s) 204.

Data storage device 206 may be for example, a random access memory (RAM), read-only memory (ROM), and/or a storage device, for example, non-volatile memory, magnetic media, semiconductor memory devices, hard drive, removable storage, and optical media (e.g., DVD, CD-ROM).

Computing device 202 may include an imaging interface 210 for communicating with one or more imaging modalities 211 that acquire a dataset of imaging data of a patient, optionally, before the intervention begins. Such an image (a/k/a pre-acquired image) may be 2D, 3D, or 4D (where one of the dimensions may be time), but as it is many times a 3D image, it is referred to below as 3D image. Examples of imaging modalities include anatomical imaging modalities, and functional imaging modalities, for example: computer tomography (CT) machine, an ultrasound machine (US), a nuclear magnetic resonance (NM) machine, a single photon emission computed tomography (SPECT) machine, a magnetic resonance imaging (MRI) machine. Optionally, imaging modality 211 acquires three dimensional (3D) data and/or 2D data and/or 4D data. In some embodiments, the connection between imaging modality 211 and the computing device 202 may be via data transfer. For example, image data from the imaging modality may be downloaded to a portable memory device (e.g., disk on key), and interface 210 may be a disk-on-key socket, allowing to upload the image data, for example, to data repository 208. In some embodiments, no pre-acquired 3D image is used. In some such embodiments, imaging interface 210 and imaging modality 211 may be omitted, and the 3D image is dynamically computed based on data received through other channels of system 200. The image may be dynamically computed based on location data of the catheter, for example, as described herein. The location data may include, for example, electrical readings, similar to those processed by the classifier to identify the intervention target region(s). Alternatively or additionally, the location data may include transformations of the electrical readings (e.g., a transformation designed to transform the readings to locations), and/or other location measurements (e.g., electrical readings of a different type, readings of magnetic sensors, etc.).

The image may be dynamically computed according to location data of the catheter, for example, based on an analysis of electrical readings of pad-electrodes 228 acquired via a pad-electrode interface 226. Pad-electrodes 228 are positioned externally to the body of the patient (e.g., on the skin of the patient, and/or in the bed supporting the patient during the intervention), and generate electrical fields. Electrical signals based on the electrical fields are used to estimate the position of catheter 216 within the organ. The voltage the pad-electrodes 228 generate is measured by electrodes on the catheter and processed to compute the 3D image of the portion of the organ. The 3D image may be computed based on an analysis of signals obtained by catheter navigation system 236, optionally a non-fluoroscopic navigation system, optionally, an impedance measurement based system. Catheter navigation system 236 may be in communication with computing device 202 via a navigation interface 234, for example, one or more of: a wire connection, a wireless connection, a software interface (e.g., SDK, API), a virtual interface, a network interface, and a local bus. Catheter navigation system 236 may be implemented, for example, as code locally stored on computing device 202, a mechanism designed to move the catheter inside the body automatically (based on the code) semi-automatically and/or manually, and/or code running on an external server.

Computing device 202 may include an output interface 230 for communicating with a display 232, for example, a screen or a touch screen. Optionally, indications of treatment instructions identified by classifier code 201A are displayed within a presentation of the 3D image, for example, the 3D image is displayed on display 232, with a marking indicating on the displayed image the location of the identified target intervention region(s). For example, a distinct color (e.g., yellow, bright green) marking on the 3D image may indicate the identified target intervention region. In another example, a border delineates the intervention target region. The intervention target region may be color coded, for example, with the color green. Another distinctly colored region (e.g., red) may indicate an identified non-intervention region which is to be avoided (i.e., treatment in that region is prohibited). The locations of intra-body electrodes 214, or of catheter 216 that carries them may be marked on the image, for example, by a dot, a star, a rod, and/or other icons, optionally with another distinct color and/or shape.

Computing device 202 may include an electrode interface 212 for communicating with one or more electrodes 214 located on a distal end portion of catheter 216 designed for intra-body navigation, for example, an electrophysiology (EP) ablation catheter, and/or other ablation catheter (e.g., chemical ablation or injection catheter). Catheter 216 may be Lasso® catheter by Biosense Webster. In some embodiments, catheter 216 may include 2-20 electrodes; e.g., 4 electrodes. The electrodes may be arranged on a straight, non-deflectable line. Optionally, the catheter may include a single tip electrode and three ring electrodes. Exemplary types of catheters 216 include: steerable, Lasso (a trademark of Biosense), non-irrigated, and irrigated.

Exemplary electrode configurations of catheter 216 include: 4 electrode ablation catheters with 1 RF electrode, 4-10 electrode single line diagnostic catheter (e.g., His, Decapole, Lasso, and the like), catheter with phased array of RF electrodes, and/or microelectrodes. Exemplary catheters 216 include: basket, Penta Ray, and 20 electrode diagnostic.

Catheter 216 may include one or more contact sensors for identifying contact between the respective contact sensor and the portion of the organ (e.g., an inner wall of a lumen within which catheter 216 is located, for example the inner wall of a chamber of a heart). The contract sensors may be implemented as dedicated contact sensors (e.g., that measure contract based on force) and/or electrode(s) 214 may serve a contact sensor function (e.g., contact may be identified by a change in impedance, voltage, and/or other electrical reading).

In one example, the electrodes perform the ablation, and sense the electrical field and/or impedance of tissue (which are analyzed by classifier(s) 201(A) for identifying of the intervention target region by the classifier(s)).

Optionally, computing device 202 includes a network interface 218, for communicating with server 222 over a network 220, for example, to obtain the pre-acquired 3D image, obtain an updated version of classifier 201A, transmit data collected from the current intervention procedure for updating the classifier, and/or access classifier 201A stored on server 222 (e.g., transmit the electrical readings via a software interface to server 222 for central processing by classifier 201A).

Optionally, a user interface 224 is in communication with computing device 202. User interface 224 may include a mechanism for the user to enter data, for example, a touch screen, a mouse, a keyboard, and/or a microphone with voice recognition software. The user may enter data via a GUI presented on display 232, where the GUI acts as user interface 224, for example, multiple intervention target regions (each identified according to different criteria) are presented on the 3D image for selection of one of the target regions by touching the corresponding location on the screen. The different criteria may reflect different specialist physicians that the instructions are designed to emulate, protocols used at different clinics, or the like

Optionally, computing device 202 includes a connector interface 242 that communicates with a connector 240 connecting to catheter 216 (e.g., RF ablation catheter, injection catheter). Connector 240 may be used, for example, to transmit control signals to catheter 216 to control administration of an intervention medical procedure, for example, control the RF ablation electrodes for an ablation procedure.

It is noted that one or more interfaces 210, 218, 212, 226, 230, 234, 242 may be implemented, for example, as a physical interface (e.g., cable interface, wireless interface, network interface), and/or as a virtual interface (e.g., API, SDK). The interfaces may each be implemented separately, or multiple (e.g., a group or all) interfaces may be implemented as a single interface.

Processor 204 may be coupled to one or more of data storage device 206, data repository 208, and interfaces 210, 218, 212, 226, 230, 234, 242.

Optionally, computing device 202 includes a data repository 208, for example, for storing: the 3D image, the received electrical reading, and/or other data (such as: health record of a patient). The data, wholly or partially, may be displayed to a user (e.g., physician) before, during and/or after the procedure. Data repository 208 may be implemented as, for example, a memory, a local hard-drive, a removable storage device, an optical disk, a storage device, and/or as a remote server and/or computing cloud (e.g., accessed using a network connection).

It is noted that computing device 202 may include one or more of the following components: processor(s) 204, data storage device 206, data repository 208, and interfaces 210, 218, 212, 226, 220, 234, 242, for example, as a stand-alone computer, as a hardware card (or chip) implemented within a current computer (e.g., catheterization laboratory computer), and/or as a computer program product loaded within the current computer.

Referring now back to FIG. 1A, it is noted that acts 104-110 include identification of instructions for treatment of the intervention target region(s), which are executed as part of the planning of the intervention procedure prior to the intervention part of the procedure, for example, after the catheter is inside the heart of the patient, but before ablation begins. No intervention treatment (e.g., ablation of tissue) necessarily occurs during acts 104-110. Acts 114-122 include adjustment of the instructions for treatment of the identified intervention target region, and are executed during the intervention part of the procedure (e.g., ablation of the tissue). It is also to be noted that some embodiments of the invention include the pre-intervention procedure alone. For example, a physician may use system 200 to identify for him initial, pre-interventional, treatment instructions, and then continue operating without further use of the system.

At 102, one or more classifiers 201A are trained, for example, as described with reference to FIG. 1B. In some embodiments, the training of the classifier may not form part of the procedure, but rather be provided in advance. The trained classifiers represent expert knowledge of one or more physicians that have performed on sample patients procedures similar to the procedure being performed on the target patient by the current user physician.

The current user physician may select the classifier from a set of classifiers. The classifiers in the set may differ from each other, for example, in the procedure they are trained to guide, in characteristics of the sample patients used for training the classifiers (e.g., demographic characteristics or the patients and/or clinical characteristics of the patients), characteristics of the equipment available to the physicians for training the classifiers (e.g., what catheters were used) and in characteristics of the physicians that trained the classifier (e.g., their clinical approach). In some embodiments, the user is provided with full data on each classifier in the set (e.g., what clinical approach was used for the training, what were the demographic characteristics of the patients, etc.), and may select a classifier from the set based on such available data.

At 104 electrical readings are received by electrodes 214 located on a distal end portion of catheter 216, located within the region of the organ of the target patient.

The electrical readings are received during an initial phase of the procedure, before treatment has begun. The treating physician navigates the catheter within the portion of the organ, collecting electrical readings from different regions of the organ portion via the electrodes on the distal portion of the catheter.

Optionally, multiple electrical readings are received, each from a different electrode 214 mounted on catheter 216. Optionally electrical readings by different electrodes are performed simultaneously and/or are received simultaneously (e.g., within a tolerance requirement, which may represent an insignificant amount of time. For example, an amount of time during which the catheter does not move significantly, so readings received simultaneously may be all attributed to the same location of the catheter).

The different electrodes 214 mounted on the catheter 216 are optionally mounted along a longitudinal axis of the catheter at a distal, rigid, end region of the catheter. Some of the electrodes (or all of them) are optionally mounted along a rigid portion of the distal end region of the catheter.

For example, using an EP (electrophysiology) catheter 216 having a single tip electrode and 3 ring electrodes, the tip electrode may be used, in addition to one or more of the ring electrodes.

The electrical readings may be generated by one or more of the following exemplary implementations: patch electrodes 228 located externally to the target individual that apply multiple alternating currents each at a respective frequency; additional electrodes on an additional intra-body catheter (i.e., additional to catheter 216) located in proximity to electrodes 214 that obtain the electrical readings, and/or additional electrodes on an additional intra-body catheter located in a predefined anatomical region, for example, within the coronary sinus as described with reference to U.S. Provisional Patent Application No. 62/449,055 “CORONARY SINUS-BASED ELECTROMAGNETIC MAPPING” by the same assignee and common inventors.

Exemplary electrical readings include measurements of one or more of the following: voltage, impedance, endocardial electrical activity, electrical activity, dielectric property, S-parameters, and combinations of the aforementioned. It is noted that under the assumption of a constant current, differences in voltage may be translated to differences in impedance. Impedance is not necessarily measured directly and absolutely.

Electrical readings may be obtained by respective electrodes 214 located on the distal end portion of catheter 216, located within the portion of the organ, for example, within the heart.

The electrical readings may include impedance electrical readings indicative of impedance of tissue touching the electrodes when the impedance electrical readings are read.

The electrical readings may be generated according to voltage readings relative to patches positioned outside the body of the patient, where the patches provide a coordinate system that is fixed in relation to the body of the patient. Optionally a set of 3 pairs of patches 228 are used, through which a low current in three distinct frequencies is applied. The 3 pairs of patches 228 are positioned to correspond to three axes, X, Y, and Z. In some embodiments, the three axes are orthogonal to each other, but in some embodiments orthogonality is compromised, in favor of, e.g., comfortable attachment of the patches to the patient's skin. The measured potentials difference, Vx, between one patch 228 and electrode 214 inside the patient's body indicates the position of the electrode 214 along the “X” axis. Optionally, Vx, Vy, and Vz are each monotonic functions along their respective axis. It is noted that impedance may be used to indicate proximity to the inner blood vessel wall, and/or proximity to the pulmonary veins.

Optionally, 3D image of the portion of the organ is reconstructed based on the electrical readings or the transformation thereof.

Systems and methods of body cavity reconstruction and/or navigation are described in International Patent Application No. PCT IB2018/050192 to the Applicant, filed Jan. 12, 2018; and in International Patent Application No. PCT IB2017/056616 to the Applicant, filed Oct. 25, 2017; the contents of which are incorporated herein by reference in their entirety. Building, for example, on descriptions in those applications, the current inventors have found that the combined use of locally calibrating spatial constraints and/or coherence constraints can be used in some embodiments of the present invention. Locally calibrating spatial constraints, in some embodiments, are provided by any parameters which constrain how different positions from which voltage measurements are obtained relate to one another. Coherence constraints, in some embodiments, constrain the spatial frequency of the main components of a transformation transforming measured values to position estimates. For example, the combined use of locally calibrating spatial constraints and coherence constraints may be for reconstruction of the image described herein, optionally the 3D image. Herein, the terms “constraint”, “constrain”, and “constraining” are used to refer to indications providing position-related information, and/or to the use of such indications by a computer-implemented algorithm, e.g., to create a reconstruction and/or locate a position within a reconstruction. In some embodiments, constraints are used in the particular context of an algorithmically derived transformation from a set of measurements taken in some physical space, to a set of positions (in that physical space) that the measurements are determined to correspond to—without relying on knowing the correct set of positions in advance. The constraints constrain how the measured properties are transformed to positions in physical space. The algorithmic derivation of the transformation, in some embodiments, expresses the constraints as cost functions (also referred to herein as error functions or penalty functions, with “more error” “more cost” or “more penalty” being understood as describing the relative value assigned to the cost functions of transformations which are relatively less satisfactory). The more the constraint is violated, the greater the cost (error, or penalty). The algorithmic derivation of the transformation, in some embodiments, seeks a transformation that minimizes (relative to other candidate transformations) the cost function. It is to be understood that constraints and constraining are not necessarily absolute. For example, constraints may be partially satisfied, optionally as measured by an appropriate weighting function (which may adjust the relative importance of different cost functions); and/or constraining may comprise reducing differences in a result relative to a constraint, e.g., by reducing the output of a cost function.

Use of locally calibrating spatial constraints, in some embodiments, optionally comprises the use of multi-dimensional scaling methods (MDS), which allow conversion of measurement distances (in whatever suitable metric, e.g., Euclidean distance or geodesic distance) into a mathematical space placing such measurements in a way that preserves those distances. The mathematical space does not necessarily, correspond to a 3-D volume. In some embodiments, additional dimensions such as heartbeat and/or respiratory phase are taken into account, allowing, e.g., construction of a phase/position space to which measurements are localized.

In some embodiments, known distances used as constraints during reconstruction include distances between electromagnetic field generating electrodes. These electrodes may also be electrodes positioned along a catheter with known spacing between them. These known distances are also referred to herein as “local calibration information”: e.g., when each of a pair of electrodes, fixed at a known distance from each other, makes a measurement of an electromagnetic field at about the same time, the known distance between them optionally calibrates the electrical field gradient between their particular measuring positions.

The problem of estimating a catheter position based on electromagnetic field measurements may be understood as the problem of finding a suitable transform to convert electromagnetic field measurements into positions (also referred to herein as transform). Considering either local calibration alone or coherence alone, there may be a plurality of transforms T that transform electrical readings to locations (i.e., transforms T(X) producing estimated positions Y′). However, some of these may fail to sufficiently satisfy Y′≈Y (that is, many possible reconstructions Y′ wouldn't look much like the reality Y). Coherence doesn't necessarily provide scale, for example, while distance constraints alone are vulnerable to cumulative distortions from measurement noise. In some embodiments, local calibration (e.g., MDS results) and coherence are combined to find a transform T based on minimization of suitably weighted joint error (or cost) in satisfying both the coherence condition and local spatial constraints. The less a condition is satisfied by some transform, the more error (cost) that condition is said to generate.

In addition to describing methods using combined local calibration and coherence, International Patent Application No. PCT IB2018/050192 describes optional use of numerous sources of additional information useful to guide reconstruction of images. Any of these sources is optionally used in some embodiments described herein. For example, in some embodiments, the additional information comprises known anatomical data. Optionally, the anatomical data is complete and fairly detailed, such as from segmentations of MRI or CT data (of the patient and/or of atlas information, optionally atlas information matched to patient characteristics such as age, weight, sex, and the like). Optionally, the anatomical data is partial; for example, comprising specifications of relative distances between anatomical landmarks. In some embodiments, a transform may be constrained to transfer measurements taken at the anatomical landmarks to positions distanced from each other by a distance known (from the anatomical data) to exist between the landmarks.

In some embodiments, landmarks are identified by their effect on movement of the probe itself (e.g., the probe's movement while partially inserted to a pulmonary vein root is limited by the circumference of the vein). Optionally, another method of identifying a landmark is used, for example, based on characteristic dielectric and/or electrical conduction properties in the vicinity of the landmark.

In some embodiments, maps of how the measurement values are expected to distribute in space (at least approximately) are used as constraints. For the case of voltage-guided navigation techniques, this can be based, for example, on simulations of electrical field voltages in space, wherein the simulations may incorporate descriptions of electrode configurations and/or body tissue dielectric properties.

It is noted that electrical fields may vary as a result of phasic motions such as heartbeat and/or respiration. International Patent Application No. PCT IB2018/050192 describes optional methods of introducing corrections for such phasic motions which are optionally used in conjunction with some embodiments of the present invention.

The electrical readings may include and/or denote position electrical readings indicative of anatomical position of the electrode(s) when the position electrical readings are read. Each anatomical position is associated with an anatomical structure, for example a specific vein, a specific artery, or a specific anatomical feature of tissue. For example, the position electrical readings may be analyzed to identify a signature electrical pattern association with a certain anatomical structure, for example, a certain heart valve, coronary sinus, or other anatomical structure. Each position electrical reading may be computed according to a reference to a coordinate system. The coordinate system may be fixed relative to the body of the patient, for example, a three dimensional space within which the organ is located. The external coordinate system may be defined by voltage readings obtained by the electrodes relative to the pad electrodes 228 located on the body of the patient, as described herein.

The electrical readings may include cardiac-phased electrical reading. Each cardiac-phased electrical reading includes an electrical reading associated with an indication as to where on a cardiac cycle the electrical reading was obtained. For example, the electrical reading may include an impedance reading and an associated indication of where on an electrocardiogram the impedance reading was obtained. The portions of the electrocardiogram represent different parts of the cardiac cycle.

The electrical readings may include respiratory-phased electrical reading. Each respiratory-phased electrical reading includes an electrical readings associated with an indication as to where on a respiratory cycle the electrical reading was obtained. For example, the electrical reading may include an impedance reading and an associated indication of where on a signal denoting the respiratory cycle the impedance reading was obtained. The portions of the signal denoting the respiratory cycle represent different parts of the respiratory cycle.

Optionally, each (or a subset) of the electrical readings is both respiratory-phased and cardiac-phased.

The electrical readings may include electrogrammed electrical readings, associated with an electrogram obtained by one or more of the electrodes 214 when the position electrical readings are obtained, so that each electrogrammed reading may be associated with a corresponding anatomical position.

Optionally, the electrical readings are generated by application of multiple alternating currents each at a respective frequency by respective electrodes. The electrodes may be, for example, pad electrodes 228, catheter electrodes 214, or other electrodes, for example, electrodes on a catheter other than catheter 226, as mentioned above.

Optionally, the received electrical reading(s) includes a reading of electrical impedance and/or another dielectric property. A dielectric property includes certain measured and/or inferred electrical properties of a material relating to the material's dielectric permittivity. Such electrical properties optionally include, for example, conductivity, impedance, resistivity, capacitance, inductance, and/or relative permittivity. Optionally, dielectric properties of a material are measured and/or inferred relative to the influence of the material on signals measured from electrical circuits and/or on an applied electric field. Measurements are optionally relative to one or more particular circuits, circuit components, frequencies and/or currents. The material whose dielectric properties may be inferred according to some embodiments may be a wall of an organ, for example, an inner wall of a heart chamber.

The electrical readings and/or transformation thereof may be stored as one or more matrices of S11 and S12 (e.g., Sij) of the electrodes at different frequencies. For example, each electrode may receive from a current source an alternating current of a distinct frequency, and transit a signal at this frequency. In addition, in some embodiments, each electrode may read voltages at each of the frequencies transmitted by the reading electrode and by the other electrodes. A ratio between the power transmitted by an electrode and the power received by the same electrode may be referred to herein as Sii. A ratio between the power transmitted by one electrode and received by another may be referred to herein as Sij. The transmitting electrode may be identified at the receiving side based on the frequencies of the signals received.

Optionally, each (or subset) of the electrical readings is associated with a time stamp indicative of the time at which the respective electrical reading was obtained by the electrodes 214. Electrical readings having time stamps within defined time windows indicative of an approximate simultaneous time of acquisition are clustered, for example, electrical readings obtained within about 0.01, 0.02, 0.05, 0.1, or 0.5, or 1, second, or other value.

Optionally, the electrical readings are mapped (e.g., registered) to a 3D image of the portion of the organ, for example, based on CT and/or MRI image data of a CT and/or MRI image(s) of the target individual. The 3D image of the portion of the organ including the mapped electrical readings may be presented on a display (e.g., display 332). For example, dots (or other marking) on the 3D image correspond to the anatomical location where the electrodes performed (or are preforming) the electrical reading. The mapping of the electrical readings to the 3D image may be performed by existing code executed by one or more hardware processors, for example, as described with reference to International Application No. IB2017/056616, “SYSTEMS AND METHODS FOR REGISTRATION OF INTRA-BODY ELECTRICAL READINGS WITH A PRE-ACQUIRED THREE DIMENSIONAL IMAGE”, incorporated herein by reference in its entirety.

Optionally, anatomical landmarks are identified based on the electrical readings, for example, as described with reference to U.S. Provisional Patent Application No. 62/504,339 “PROPERTY- AND POSITION-BASED CATHETER PROBE TARGET IDENTIFICATION”, by the same assignee and common inventors.

In some embodiments, the 3D image is computed based on position electrical readings, and electrical readings of another type (e.g., electrograms) are also used for planning the intervention or updating the planning. In the following, the electrical readings used for obtaining the 3D image or marking it with an indication of intervention target region are referred to as electrical readings of the second type, and the other electrical readings are referred to as electrical readings of the first type.

In some embodiments, the electrical readings of the first type are associated with locations at which the electrical readings of the second type were obtained by the electrodes. The locations of the electrical readings of the first type are mapped to corresponding locations of the 3D image.

The electrical readings (both of first and second type) may be registered with the 3D image based on measurements (e.g., other and/or the same electrical readings) representing the position of electrode(s) 214 of catheter 216, optionally based on measured potential (e.g., voltage) relative to body surface electrodes (also referred to herein as patches or pad electrodes 228) located outside the body of the patient, for example, on the skin of the patient.

At 106, one or more additional features are received. The additional features represent additional data that is fed into the classifier with the goal of improving the relevancy and/or accuracy of the identification of the instructions for treatment of intervention target region. The additional features may allow personalization of the classification according to the target individual being treated. Receiving the additional features is merely an optional feature of the present invention, as the classifier may be trained to identify treatment instructions without using them, for example, based on electrical readings alone.

Optionally, the additional features include features extracted from a profile of the target patient. The profile of the target patient may be stored within the electronic medical record (EMR) of the patient, and/or within another dataset. Feature extracted from the profile may denote clinical features of the patient, and/or medical history of the patient associated with intervention procedures. Exemplary features extracted from the profile of the target patient include one or more of, and/or combinations of: gender, age, height, weight, body mass index (BMI), clinical data (e.g., smoking history, previous interventional procedures, congenital abnormalities, anatomical variations, genetics), and family history of interventional procedure (and/or medical conditions that warranted an interventional procedure)), and demographic data. The features extracted from the profile are used by the classifier to personalize the identification of instructions for treatment of the intervention target regions according to other patients that are similar to the target patient in terms of the features extracted from the profile. It is assumed that improved, more accuracy, and/or more relevant results are obtained when the intervention target regions are identified according to similar patients.

Optionally, the additional features include additional tissue properties. The additional tissue properties may be obtained by one or more sensors designed to measure the respective tissue property, for example, a laser emitter and a receiver to measure reflectance. The additional tissue properties may be obtained from previous measurements and/or from the patient electronic medical record, for example, the presence of detected scars, and/or tissue thickness measured from an earlier CT scan. The tissue properties may be measured at the same location (or in near proximity to) as where electrodes 214 performed the electrical readings. The sensor(s) may be located within the portion of the organ where electrodes 214 that measure the electrical readings are located. The tissue properties may be measured as a different location where electrodes 214 performed the electrical reading, and may be associated with data indicative of the absolute and/or relative location at which the tissue properties were measured. An example of a relative location includes a vector relative to the location where a certain electrical reading was made. An example of an absolute location includes an anatomical location independent of location where other electrical readings (e.g., within the coronary sinus). Exemplary additional tissue properties include: molecular structure, IR reflectance, NIR reflectance, Ho Yag reflectance, Ph, ion concentration, reactance, tissue thickness, scars and combinations of the aforementioned. The tissue properties are used by the classifier to identify the instructions for treatment of intervention target regions based on similarities in tissue properties of previously treated patients. It is assumed that improved, more accuracy, and/or more relevant results are obtained when the intervention target regions are identified according to similar tissue properties, since for example, an intervention target region is more likely to be found at one type of tissue property rather than another type of tissue property.

Additional features may include an anatomical relative and/or absolute distance to a defined fiducial (e.g., measured in millimeters), for example, the coronary sinus, dimensions of the tissue (e.g., tissue thickness), and/or absolute and/or relative location in a functional domain (e.g., measured in millivolts or other functional measures) for example, voltage, potential, impedance, and/or other tissue qualities. The distance relative to a defined fiducial is fed into the classifier for further improving the accuracy of identifying the intervention target region.

Additional features may be based on tissue properties (e.g., properties measured prior to the treatment and tissue properties measured during treatment and/or post-treatment) to compute the effect of the treatment on the tissue region.

Alternatively or additionally, the additional features include a hardware type of the treatment device for performing the treatment. The hardware type may include a hardware profile of one or more parameters of the hardware of the treatment device, for example, manufacturer, device model, ranges of settings, and treatment modality (e.g., RF, cryo). The additional features may include settings of the treatment device. The settings may be included as parameters of the hardware profile. Exemplary settings include, for example, direction of application of energy by the treatment device, power intensity of the applied energy, time of the applied energy, and/or pattern of the applied energy.

At 108, the classifier identifies instructions for treatment of a region in the portion of the organ as an intervention target region. The classifier identifies the instructions for treatment of the region based on electrical readings and/or a transformation thereof and optionally the additional features (e.g., as described with reference to act 106) previously associated with treatment of intervention target regions in the portion of the organ of other patients.

The classification may be performed according to training based on procedures performed by a certain physician, and/or based on a set of physicians following a common treatment approach. The user may select which physician (from a list of possible physicians) to use for classification and/or select which treatment approach (from a list of possible treatment approaches) to use for classification, and/or code may automatically select the physician and/or treatment approach to use for classification, for example, according to the physician that is considered an expert in performing the procedure based on an analysis of the procedure data (e.g., entered by a user and/or automatically extracted from a procedure planning report).

Optionally, the instructions include a rational associated with the identified intervention target region. The rational is presented to the user (e.g., presented on a display, played as an audio message on speakers). The rational has been previously provided by the physician during training of the classifier, for example, a recording of the physician during training and/or a message typed in by the physician during training. The rational may be presented when the classifier identifies a close correlation between the current electrical readings and/or transformation thereof and the training data, indicating that the user physician is being closely guided by a previously observed scenario. Alternatively, the rational may be presented when the classifier identifies a not very significant correlation, indicating that the user physician is unsure of what to do next, or is moving away from a correct intervention path. An example of a rational: “I'm looking for a place at the back wall of the LA, where the spectrogram is distinctly much higher from its surrounding”. The user may select or decline the instructions for treatment of the identified intervention target region in view of the rational.

In one example, when a new patient is being treated by a user that selects performing identification of instructions for treatment of intervention treatment regions according to a certain physician, the user maps the back wall of the heart by collecting electrical readings. The classifier identifies regions that have similar characteristics and/or similar anatomical locations as the regions mapped by the user (e.g., distinct spectrogram), and marks the region as the intervention treatment region based on corresponding markings manually designated by the certain physician on other sample patients.

In another example, the classifier may select intervention target regions only on a right side of a ridge of tissue, and never on the left side of the ridge, according to learned behavior of the certain physician. For example, the classifier has been trained according to behavior of the certain physician, which only designated intervention target regions on the right side, and/or designated non-intervention target regions on the left side.

It is noted that the user may override the behavior of the selected physician (e.g., by making a suitable selection within the GUI), and/or adjust the classification according to the selected physician (e.g., by defining a set of rules, or making a selection from a list). For example, the user may override the constraints on the classifier by the selected physician to only treat the right side, by instructing the classifier to select intervention target regions on the left side. In another example, the user may define constraints on the classifier according to demographics of the patient. For example, to only identify intervention target regions on the right side for women.

The identification of the instructions for treatment of intervention target region may be performed according to a weighted combination of the different types of electrical readings (e.g., impedance, anatomical position, cardiac-phased, respiratory-phased, electrogrammed) and/or additional features. The higher the resemblance of the weighted combination to corresponding values of previously associated treatments of intervention target regions, the higher the probability that the instructions for treatment of region is identified. The weights may be automatically computed, for example, by a neural network, and/or manually defined, for example, by an expert physician according to clinical knowledge of what is most relevant.

The classifier may output the probability that the instructions for treatment of the intervention target region are correct, optionally according to the resemblance of the weighted combination.

In some embodiments, the electrical readings are first converted into instructions for treatment of the intervention target regions, and then the instructions for treatment of the intervention target regions are marked on the image. The 3D image may be reconstructed from the electrical readings and/or transformations thereof, as described herein. The intervention target region is marked on the reconstructed 3D image according to image regions that correspond to the electrical readings and/or transformations thereof identified as intervention target region(s). Alternatively, in other embodiments, the image is first reconstructed from the electrical readings, and the instructions for treatment of intervention target region are identified according to the regions of the image. The image portion(s) of the 3D image are identified as instructions for treatment of target intervention readings according to electrical readings and/or transformations thereof mapped to the image portions.

In some embodiments, the electrical readings and/or transformations thereof are identified by classifier(s) 210A as instructions for avoidance treatment of non-intervention target regions, indicative of a region in which intervention is prohibited. The non-intervention target regions denote tissue locations that should be avoided, for example, sensitive locations that should not be ablated. The classifier identifies the instructions for avoidance of treatment of non-intervention target region(s) based on observed associations between previously analyzed electrical readings and/or transformations thereof and avoidance of treatment of regions in the portion of the organ previously identified as non-intervention target regions. The regions previously identified as non-intervention regions may be explicitly identified as non-intervention target regions (e.g., by a user manually marking the region as non-intervention) and/or may be identified implicitly by an analysis of electrical readings and/or transformations thereof that have not been indicated as intervention regions for multiple patients. Consistent avoidance of intervention at certain region(s) in multiple patients may be assumed to be indicative of non-intervention target regions. The consistent avoidance may be identified according to the type of intervention being performed, since treatment at a certain location may be desirable for one type of intervention while the same location is to be avoided when performing another type of intervention. For example, one part of a heart chamber that includes a certain type of neural tissue is suitable for ablation treatment but not suitable for a needle puncture. Another part of the heart chamber that connects to an adjacent chamber (e.g., fossa ovalis) is suitable for needle puncture, but unsuitable for ablation treatment.

Optionally, the classifier may identify the instructions for treatment of the region as the intervention target region based on impedance electrical readings previously associated with treatment of intervention target regions in the portion of the organ of other patients.

Optionally, the classifier identifies the instructions for treatment of intervention target region according to clusters of electrical readings or a transformation thereof previously associated with treatments of intervention target regions in the portion of the organ of other patients. The cluster may include electrical readings recorded within a time window, for example, about 0.1, or 0.3, or 0.5, or 1, or 3 seconds. The cluster of electrical readings within the time window may represent electrical readings recorded approximately simultaneously, and therefore treated as a single measurement.

Alternatively or additionally, the classifier(s) identifies the instructions for treatment of the region as the intervention target region based on previously analyzed one or more tissue properties and regions in the portion of the organ previously treated when identified as intervention target regions.

Alternatively or additionally, the classifier(s) identifies the instructions for treatment of the region as the intervention target region based on position electrical readings (indicative of anatomical position of the electrode(s) when the position electrical readings are read) previously associated with treatment of intervention target regions in the portion of the organ of other patients.

Alternatively or additionally, the classifier(s) identifies the instructions for treatment of the region as the intervention target region based on anatomical positions previously associated with treatment of intervention target regions in the portion of the organ of other patients.

Alternatively or additionally, the classifier(s) identifies the instructions for treatment of the region as the intervention target region based on combinations of anatomical positions and impedance electrical readings. The combinations being previously associated with treatment of intervention target regions in the portion of the organ of other patients.

Alternatively or additionally, the classifier(s) identifies the instructions for treatment of the region as the intervention target region based on cardiac-phased electrical readings, previously associated with treatment of intervention target regions in the portion of the organ of other patients.

Alternatively or additionally, the classifier(s) identifies the instructions for treatment of the region as the intervention target region based on respiratory-phased electrical readings, previously associated with treatment of intervention target regions in the portion of the organ of other patients.

Alternatively or additionally, the classifier(s) identifies the instructions for treatment of the region as the intervention target region based on electrogrammed electrical readings, previously associated with treatment of intervention target regions in the portion of the organ of other patients.

Alternatively or additionally, the classifier(s) identifies the instructions for treatment of the region as the intervention target region based on resemblance between the profile of the patient and profiles of the other patients. For example, the classifier considers data from other patients having profiles that correlate to the profile of the target patient. For example, other patients that have one or more (or combination) of the following similar parameters: gender, weight, height, BMI, clinical data, demographic data, smoking history, history of previous interventions, and similar genetics.

Alternatively or additionally, the classifier(s) identifies the instructions for treatment of the region as the intervention target region based on hardware types of the treatment device for performing the treatment, previously associated with treatment of intervention target regions in the portion of the organ of other patients.

Optionally, the instructions for treatment of the intervention target include instructions for treatment of an ablation region that is designated for treated by ablation (e.g., radiofrequency ablation, cryoablation). The ablation region may include an ablation line. One or more ablation lines may be computed.

The classifier(s) may identify instructions for treatment of multiple regions identified as intervention target regions. The instructions for treatment of multiple intervention target regions may be identified substantially simultaneously according to the received electrical readings and/or transformations thereof and/or additional data. The instructions for treatment of multiple intervention target regions may be identified sequentially, as the catheter with electrodes is moved to different locations within the organ. The instructions for treatment of the ablation region may be defined according to the multiple regions. For example, spaced apart electrical readings may be identified as spaced apart intervention target regions. The ablation region, optionally ablation line, may be defined as a region that includes the spaced apart regions, for example, a line or strip that passes through the spaced apart target regions and/or that includes a boundary that encompasses the multiple spaced apart target regions.

Optionally, the electrical readings and/or transformations thereof are classified into instructions for treatment of multiple intervention target regions, each of one or more types. Exemplary types may be based on, for example, different clinical criteria, different procedures, different treatment devices (e.g., different manufacturers, different models), different treatment modalities (e.g., RF, cryo, chemical), and different risk levels. The instructions for treatment of types of intervention target regions may be selected from a set of predefined types. Each cluster of a certain sub-set of electrical readings and/or transformations thereof is classified into one of the multiple intervention types. The clusters may overlap, with electrical readings included in one or more clusters. Optionally, the ablation region denotes an ablation region for pulmonary vein isolation (PVI) ablation.

Optionally, each intervention target type denotes a respective ablation line based on different identification criteria, for example, different electrical signal patterns and/or different parts of transformations of the electrical signals. The different identification criteria may be medically accepted criteria that provided different options for different locations of ablation lines. Exemplary criteria for an ablation line for pulmonary vein ablation are based on one set of the following criteria. Each set of the following criteria denoting a different type of ablation line for pulmonary vein ablation:
Electrical readings from the pulmonary artery and left atrium being equal according to an equality requirement (e.g., denoting the maximum difference between the electrical readings that still considers the electrical readings as being equal).
Maximum simultaneous values of the electrical readings from the pulmonary artery and left atrium according to a maximal requirement (e.g., denoting how far apart the maximum values of the electrical readings may be to still be considered simultaneous).
Presence of the left atrium electrical reading and absence of the pulmonary artery electrical reading within a presence-absence requirement (e.g., denoting the range of values of the pulmonary artery electrical readings that are considered as absent, and/or denoting the range of values of the left atrium electrical readings that are considered as present).

Optionally, the instructions for treatment of the intervention target include a location corresponding to a certain anatomical structure designated for treatment. For example, the intervention target may include the fossa Ovalis, for example, instructions for performing a trans-septal puncture at the fossa Ovalis. Instructions for avoiding treatment of a non-intervention target classification may be made for locations corresponding to tissue external to the fossa Ovalis at which a puncture is prohibited.

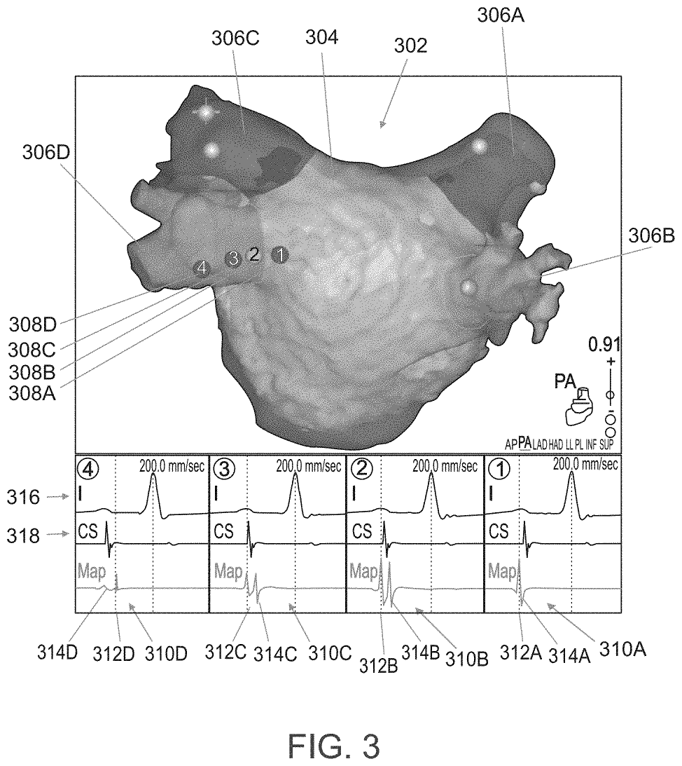

Optionally, the classifier identifies instructions for treatment of the intervention target region based on previously observed scores indicative of success of an intervention treatment applied to intervention target regions in the portion of the organ of other patients. For example, as part of the process of training the classifier (e.g., as described with reference to FIG. 3) the score is assigned to the intervention treatment performed according to the designated intervention target regions of the other patients. The score may be computed automatically (e.g., based on an analysis of the patient medical record, for example, identifying a change in diagnosis of the patient) and/or manually provided (e.g., entered by the physician). The score may be binary, for example, success or failure, or a classification category (e.g., highly successful, average success, limited success, failure), or a value indicative of success (e.g., on a scale of 0-10). The classifier may perform the identification of the instructions for treatment of the intervention target region according to maximization of a predicted likelihood of success of an intervention treatment performed according to the identified intervention target region. The predicted likelihood of success is based on the assigned score. For example, when instructions for treatment of two intervention target regions are possible candidates for the electrical readings, the classifier may select the instructions for treatment of the intervention target region associated with a higher predicted likelihood of success by selecting the instructions for treatment of the intervention target region associated with a higher score. Alternatively, both instructions for treatment of intervention target regions are presented, along with an indication of the score indicative of predicted likelihood of success, allowing the treating user physician to select between the instructions for treatment of intervention target regions. For example, one set of instructions for treatment of intervention target region may be associated with a higher success rate, but is technically more challenging and/or poses greater safety risk than another set of instructions for treatment of intervention target region with a lower success rate, which is technically easier and/or poses less safety risk.

Optionally, the instructions for treatment of the intervention target region are computed as an adjustment to manually designated instructions for treatment of intervention target region. The manually designated instructions for treatment of the intervention target region may be provided, for example, by a user via the GUI that presents the 3D image and/or location of electrical readings on the 3D image. For example, the user may manually delineate boundaries of the intervention target region on the 3D image according to the location of the electrical readings, via the GUI. In another example, the user may select the settings (e.g., intensity, direction, time) of the treatment device and enter the selected hardware type of the treatment device. Instructions for treatment of an intervention target region identified by the classifier(s) (as described herein) is correlated with the manually designated instructions for treatment of the intervention target region according to a correlation requirement (e.g., defining the tolerance of allowable difference between the regions, for example, in terms of location and/or size, and/or defining the tolerance of allowable difference between the settings of the particular type of treatment device). An adjusted to the manually designated instructions for treatment of the intervention target region that satisfies the correlation requirement is computed. The computed adjusted is designed to fit the manually designated instructions for treatment of the intervention target region to the instructions for treatment of the intervention target region identified by the classifier(s). The adjustment may be computed, for example, by computing the vector(s) that when added to the manually designated intervention target region and/or treatment device setting result in the intervention target region and/or treatment device setting identified by the classifier(s), while remaining within the correlation requirement. The adjusted portion of the instructions may be presented on the 3D image with an indication that is different than the indication of the manually designated instructions for treatment of the intervention target region. For example, the manually designated intervention target region is shown in yellow, and the portion of the manually designated intervention target region that is affected by the adjustment is shown in red, and the adjusted portion is shown in green. In another example, the manually entered device settings are shown in yellow, the amount of adjustment is shown in red, and the computed device settings are shown in green.

At 110, an indication(s) of the instructions for treatment of the region identified by the classifier as the intervention target region(s) is displayed on the 3D image of the portion of the organ.

The indication of the identified intervention target region may be marked on the 3D image as, for example, a dot (or other shape, for example, an arrow, a square, a triangle), optionally type coded (e.g., by color, and/or shape) to represent respective intervention types.

The instructions for treatment may include one or more of: a marking of the intervention target region on the 3D image, a text message presented on a display indicating how to treat the intervention target region, an animation simulating treatment of the intervention target region, a video captured of another user previously performing a treatment, and an audio recording that instructions how to treat the intervention target region.

Optionally, an indication for each of multiple instructions for treatment of intervention target regions may be generated for presentation, for example, when the classifier identifies multiple sets of instructions for treatment of possible intervention target regions. The multiple instructions for treatment of intervention target regions may be of the same type, for example, based on the same clinical criteria, and/or based on the same treatment device. Each set of instructions for treatment of intervention target region may be associated with a computed probability (as described herein). The multiple sets of instructions for treatment of intervention target regions may meet a probability requirement, for example, having a probability value above a threshold (to exclude unlikely target regions). The user may select one of the sets of instructions for treatment of intervention target regions for guiding the treatment.

The indication may be implemented as a graphical overlay positioned over the 3D image.

The indication may represent an ablation line on the 3D image for ablation, for example, an ablation ring or other topology.

When multiple sets of instructions for treatment of intervention target region of different types are identified, each set of instructions for treatment of intervention target region may be presented on the 3D image of the portion of the organ with a distinct identifier associated with each one of the types of intervention target regions. Each type of instruction set for treatment of intervention target region may represent an anatomical region for ablation based on a certain set of clinical criteria, and/or a certain treatment type such as a certain type of ablation energy (e.g., RF, cryo). The instructions for treatment of intervention target regions of different types may overlap one another. For example, each intervention target region of a first type is presented with a border delineating each region of a first color, and each intervention target region of a second type is presented with a border of another color. The simultaneous presentation of sets of instructions for treatment of intervention target regions based on the different criteria and/or treatment types allow for the physician to compare procedures performed according to the different criteria and/or treatment types, to select the most suitable one. For example, the physician may decide when looking at the simultaneous presentation of sets of instructions for treatment of intervention target regions that ablation using RF is technically easier than ablation with cryo. In another example, the physician may decide that performing the procedure according to one set of clinical criteria is safer than performing the procedure according to another set of clinical criteria.

The user may select (e.g., via the GUI) which types of instructions for treatment of intervention target regions to present. For example, a list of the types may be presented in association with a check-box. The user may click on the check-boxes to select the type(s) of instructions for treatment of intervention target regions to present. The non-selected types may be removed from presentation (or not presented initially). For example, the user may select one set of clinical criteria to exclude other instructions for treatment of intervention target regions according to other criteria, or may select two different sets of clinical criteria to compare the associated instructions for treatment of intervention target regions for selection between the two different sets of clinical criteria.

When multiple ablation lines are computed (e.g., based on different criteria), all the ablation lines may be presented on the 3D image, where each ablation line has a distinct topology over the 3D image based on the respective identification criteria. Ablation lines may overlap one another. Each ablation line may be distinctly marked, for example, by a different color. The user may select one of the ablation lines (e.g., via a graphical user interface (GUI)) to be used for performing the ablation procedure. The other ablation lines may be removed from the 3D image.

Alternatively, the user may select one or more criteria (e.g., via the GUI). The ablation lines corresponding to the selected criteria are presented on the 3D image. The ablation line corresponding to the selected criteria may be used for performing the ablation procedure.

Optionally, the instructions for treatment may include recommendations, for example, indicating from which part of the intervention target region(s) to start the treatment, settings for the treatment (e.g., time of ablation, amount of ablation energy to apply, type of probe for performing the treatment, and/or type of ablation energy). The recommendation may be presented, for example, as a set of text-based instructions presented next to the 3D image (e.g., within the GUI), as an animation video incorporating the 3D image, and/or an audio message played over a speaker(s).

The recommendations may be presented as stored audio recordings and/or video recording and/or typed message of one or more of the expert physicians that performed procedures that were used to train the classifier(s).

The indication of recommendation for proceeding in the treatment to treat the intervention target region may be dynamically computed and/or dynamically updated, as part of an iterative process (e.g., described with reference to act 124) according to the identified updated intervention target region (e.g., described with reference to act 120), for presentation in association with the 3D image and/or playing as an audio message.

Optionally, the instructions include one or more treatment modalities for application to the identified intervention target region. Exemplary treatment modalities include one or more of: probe pressure, heating, cooling, cardiac pacing, defibrillation, radiofrequency energy application, radiofrequency ablation, cryo application, cryo ablation, other energy delivery, and combinations of the aforementioned.

At 114, the intervention procedure is in progress. At least one tissue region of the organ is treated, for example, ablated. The treated tissue region(s) may include (or be) the identified intervention target regions.

The additional electrical reading(s) are obtained after tissue region(s) of the organ are treated (e.g. ablated). The additional electrical readings are obtained after at least a portion of an interventional procedure is performed according to the instructions for treatment identified by the classifier(s).

The additional electrical reading(s) obtained during the intervention may be presented on the 3D image displayed on the display, for example, as additional details in the 3D image.

At 116, optionally, one or more additional dynamic features are extracted from data affected by the intervention procedure. The one or more additional features may correspond to dynamic features that were extracted prior to the intervention, for example, received as described with reference to act 106 of FIGS. 1A-B. The post-intervention dynamic feature may be compared to the pre-intervention dynamic feature to identify the change due to the intervention. For example, the following dynamic features may be extracted from tissue region(s) at and/or in proximity to the intervention region (and/or at other regions) to compute changes due to the intervention: changes to a pressured applied by a probe, changes to effects of heating on the tissue, changes to effects of cooling on the tissues, natural changes occurring to the tissue over time (e.g., scarring), changes that correlate to the heart beat, changes that correlate to the respiratory cycle, changes when pacing is applied, changes when defibrillation is applied, changes with delivery of energy excluding RF and cryo.

At 118, the classifier(s) identifies one or more adjusted instructions for treatment of the intervention target regions according to the additional electrical reading(s) and/or transformations thereof. The adjusted instructions for treatment of the intervention target regions represent a correction of the initially identified instructions for treatment of the intervention target regions by considering the new electrical readings which were not used to identify the initial instructions for treatment of the intervention target regions, for example, because they have not been available before the intervention.

The adjusted instructions for treatment of the intervention target region may be identified by the classifier re-identifying a previously treated identified target intervention target region according to the additional electrical readings and/or transformations thereof. The adjusted instructions for treatment of the intervention target region may be identified by the classifier as treatment of a new target intervention region (which may overlap, wholly or partially, a previously identified intervention target region) according to the additional electrical readings and/or transformations thereof.

The classifier(s) identifies the adjusted instructions for treatment of the intervention target region based on observed associations between previously analyzed electrical readings and/or transformations thereof and/or additional features including electrical readings obtained after tissue(s) of the portion of the organ, which is optionally identified as target invention region(s), is treated and region(s) in the portion of the organ previously identified as updated intervention target regions after at least some treatment applied to earlier identified intervention target regions(s).

Optionally, the adjustment to the instructions for treatment of the intervention target region is computed according to an indication of a treatment region in which an intervention treatment was performed within the portion of the organ. The adjustment may be computed in real-time, as the physician is treating the intervention target region. For example, the classifier(s) may initially compute instructions for ablation of a set of ablation regions that are spaced apart from one another by defined distances. The physician ablates a first ablation region at a location that is different than the corresponding location of the first ablation region according to the instructions computed by the classifier(s). The instructions for ablation of the set of ablation regions are dynamically updated to account for the actual ablation performed, for example, by re-computing the remaining ablation regions and/or re-computing the settings of the ablation device in view of the location of the actual ablation and/or in view of the actual settings of the ablation device used to perform the ablation.

At 120, an indication of the adjusted instructions for treatment is computed for marking on the 3D image of the portion of the organ. The adjusted instructions for treatment may replace the previously identified instructions. Alternatively, the adjusted instructions for treatment is presented with a different indication that the previously identified instructions for treatment, for example, using a different color, a different font, and/or a different label icon. The adjusted instructions for treatment of the intervention target region may be overlaid on the previously identified instructions for treatment of the intervention target region. For example, the previously identified intervention target region is presented in light yellow, and the adjusted intervention target region is presented in green, and overlaid on the light yellow.

The indication of the adjusted instructions for treatment may be presented on the 3D image. The adjusted intervention instructions may be presented in addition to the original computed instructions, for example, based on a distinct color to differentiate the original instructions from the adjusted instructions. Alternatively, the original presented instructions are replaced with the adjusted instructions.

The adjusted instructions may include, for example, adjusted intervention target region. For example, based on the additional electrical readings received during the intervention, the classifier may learn that the region ablated in fact is different from the region that should have been ablated according to the instructions identified before (e.g., during act 108), and an update of the remaining ablation targets is offered by the classifier to keep emulating the training physician in view of the new situation created during the intervention.

Optionally, the adjusted instructions may include adjustments to power of ablation and/or time of application of ablation energy. The power of ablation and/or time of application of ablation energy may be initially computed before the intervention procedure and dynamically updated during the intervention procedure. The adjustments may be performed by the classifier(s) based on observed associations between previously analyzed powers of ablation and times of application of ablation energy and treatments of intervention target regions.

At 122, one or more predictions and/or estimations are computed according to the identified instructions for treatment. The predictions and/or estimations may be computed, for example, in association with and/or in parallel to one or more of acts 108-120, for example, before the intervention procedure and/or during the intervention procedure and/or dynamically updated as additional data is available. The predictions and/or estimations may be performed by the classifier(s) based on observed associations between previously analyzed values (which are being predicted and/or estimated) and treatment results. It is noted that act 122 is optional, and may be omitted in some embodiments, as there is no necessity to predict or estimate anything other than recommended treatment instructions.

Optionally, the total procedure time for performing the intervention procedure is estimated by the classifier(s) based on observations between previously analyzed procedure times for performing a similar intervention procedure on other sample patients. When the computed total estimated procedure time is identified to be significantly longer (e.g., above a threshold) than a first estimate of the procedure time, the last patient(s) of the day may be dismissed in advance based on the obtained knowledge that the procedure time(s) for patients scheduled earlier in the day are expected to take longer than originally planned for.

At 124, the features described with reference to acts 114-122 are iterated, as new electrical readings are obtained during the intervention procedure.

Reference is now made to FIG. 1B, which is a flowchart of a method of training one or more classifiers to identify instructions for treatment of the intervention target region(s), in accordance with some embodiments of the present invention. It is noted that some features of training the classifier are described with reference to FIG. 1A, for example, with reference to act 108, which describes that the classifier identifies the instructions for treatment based on electrical readings and/or a transformation thereof and/or other described features previously associated with treatment of intervention target regions in the portion of the organ of other patients.

The Classifier is trained by collecting data for many sample patients (also referred to as sample individuals). Acts 152-156 are executed for each of the sample individuals prior to the intervention procedure, as part of the planning of the intervention procedure. No intervention treatment (e.g., ablation of tissue) necessarily occurs during acts 152-156. Acts 158 and 160 are executed after the interventions end. Data from all the interventions are collected and analyzed to generate associations between treatment plans planned by the experienced physician at act 156 and measurements results the experienced physician received at 152 and the additional data the experienced physician received at 154.

At 152, for each of multiple sample individuals, electrical readings and/or transformations thereof are received, for example, as described with reference to act 104 of FIG. 1A. The electrical readings are obtained by respective electrodes 214 located on catheter 216 within a portion of an organ of the respective sample individual, as described herein. The electrical readings are mapped to or used to create the 3D image of the portion of the organ, as described herein.

At 154, features may be extracted from additional data, for example, the patient profile, as described with reference to act 106 of FIG. 1A. The additional extracted features may be associated with one or more electrical readings, but is not necessarily so associated, as explained above in relation to act 106.

At 156, for each of the multiple sample individuals, an indication of instructions for treatment of each region in portion(s) of the organ is received from the experience physician running the operation. The indication(s) denotes the treatment plan planned by the experienced physician. The instructions for treatment of the intervention target region is associated with the electrical readings based on which the plan is designed by the experienced physician and/or a transformation of these readings. The indication may be manually defined by the experienced physician or another member of the staff working with him via user interface 224, which may include the GUI. For example, the indication may include manually delineating a border through a subset of electrical readings and/or transformation thereof, a text message entered into the GUI, a video of the user performing the procedure is recorded in association with the electrical readings, and/or a link to an animation is entered into the GUI.

Alternatively or additionally, for each of the multiple sample individuals, an indication of instructions for avoidance of treatment of each region in the portion(s) of the organ denoting a non-intervention target region is received through user interface 224 (e.g., the GUI). The non-intervention target region may denote a region to avoid treatment, for example, regions where ablation is prohibited.

Optionally, the user provides a rational for the treatment instructions, for example, a rational for selection of regions as intervention target regions and/or for selection of other regions as non-intervention target regions. The rational may be provided, for example, by being manually typed via the GUI, and/or a recording of the user speaking (which may be converted into text by speech-to-text code). The rational may be stored in association with the intervention target region, for example, as metadata, as a tag, and/or as a record. The rational may be presented to the user (e.g., presented on a display, played as an audio message on speakers) when the trained classifier identifies that the readings received from a target individual are indicative of a situation similar to that under which the rational was provided by the experienced physician. An example of a rational: “I'm looking for a place at the back wall of the LA, where the spectrogram is distinctly much higher from the spectrograms in its surrounding”.

At 158, one or more clusters, each including a subset of the electrical readings may be associated with a label indicative of a type of instructions for treatment, for example, as a tag, and/or metadata. Members of the cluster may be manually marked by the user and/or automatically identified by code. Electrical readings that are not explicitly marked by the user may be implicitly marked and clustered. Alternatively or additionally, one or more clusters of electrical readings are associated with a label indicative of non-intervention target.

Optionally, each cluster represents instructions for treatment, for example, a target region to be treated, an amount of pressure to be applied by the catheter to the target region, a duration and/or power of energy application to the target region, etc.

Alternatively or additionally, the sample individuals are clustered according to anatomical variations. The indication of a certain type of anatomical variation (selected from multiple anatomical variations) may be provided, for example, manually by a user, and/or automatically by code for example, by extracting the anatomical variation from the electronic medical record of the sample individual storing pre-identified anatomical variation(s), and/or automatically by image processing of an image obtained based on the electrical readings, e.g., based on considerations of local scaling and coherency as discussed above. The anatomical variations may relate to anatomical variations of the organ in which the intervention treatment is performed. Examples of anatomical variations include: congenital deformations, absence of one or more blood vessels, additional blood vessels (e.g. fifth pulmonary vein), variation in orifice of the one or more blood cells, and shape of organs. In some embodiments, the individual clustered into clusters so that sample individual members of each cluster have a similar type of anatomical variation.

Alternatively or additionally, the sample individuals are clustered according to treating physician, and/or a treatment approach of the treating physician. For example, sample individuals of a certain expert in a certain procedure are clustered together. The classifier trained according to the sample individuals of the same physician may effectively learn the treatment approach of that physician, and guide other physicians according to the same approach. Clustering according to the same physician and/or clustering according to treatment approach by different physicians may reduce noise which occur due to mixing of approaches.

The classifier(s) is trained according to the electrical readings and/or transformation thereof of sample individual members of each cluster and associated treatments of the intervention target regions, for identifying instructions for treatment for a new target patient associated with an indication of one or more of the anatomical variations. Multiple classifiers may be trained according to the different clusters, each trained based on one of the clusters.

Optionally, each cluster denoting treatment of a respective intervention target (e.g., ablation line) is further associated with an indication (e.g., score) of a successfulness of the respective intervention target (e.g., ablation line), that is, the score may indicate how successful the procedure was found to be eventually. Procedures found to fail, even if carried out by an experienced physician, are optionally taken out of the training sample once the failure is found out (e.g., at a later examination of the patient). For example, the indication may be a binary variable indicative of success or failure, or a discrete number scale and/or continuous value (e.g., value from 1-10, or a percent) representing the extent of success where 0 denotes complete failure and 100 denotes total success. The indication of success of treatment (or lack of success) may be provided (and updated) post treatment, for example, on the same day, or several weeks, months, or years after the treatment. The indication may be automatically identified (e.g., by code extracting data from the patient medical record) and/or manually provided.

Each type of intervention target region (e.g., ablation line) may be manually marked on the 3D image by the user, for example, by selecting the type from a list for each electrical reading member of each intervention target region type, and/or marking a border around a cluster of electrical readings with a distinct color indicative of type and/or attaching a virtual tag indicative of type, optionally with a GUI. It is noted that locations from where electrical readings are obtained may be presented on the 3D image, for example, as dots, stars, icons, or other representations. Each intervention target region (e.g., ablation line) may include regions of the portion of the organ external to regions where the electrical readings were obtained, for example, between the locations where the electrical readings were obtained. Each indication of a respective intervention type (e.g. ablation line type) has a different topology on the 3D image. Indications may overlap each other. The indication may be presented as a border encompassing the electrical readings associated with the cluster of the certain intervention target type.

Optionally, each of the intervention types represents a respective ablation line type based on different criteria for identified each respective ablation line. For example, each one of the criteria described with reference to act 108 defines one ablation line type.

At 160, one or more classifiers are trained according to the electrical readings and/or transformations thereof and/or additional features, and the label(s) associated with each defined cluster of the electrical readings indicative of the intervention target region(s), collected for each of the sample individuals. Optionally, the classifier(s) are generated in view of the additional extracted features.

The classifier(s) are trained to identify, for a new target patient, an intervention target region based on electrical readings obtained for the new target patient or a transformation thereof, for presentation on a 3D image of the organ of the new target patient an indication of the identified intervention target region.

Optionally, the classifier is generated for computing the instructions for treatment of the intervention target region according to maximization of a predicted likelihood of a successful intervention treatment procedure outcome, based on the indication (e.g., score) of the successful treatment outcomes and/or non-successful treatment outcomes associated with each cluster of electrical readings of the sample individuals. The classifier may compute the instructions for treatment of the intervention target region associated with the highest computed prediction of success of treatment following the computed intervention target.

Alternatively or additionally, the generated classifier outputs a prediction of likelihood of success of treatment following the computed instructions for treatment of the intervention target, for example, as a probability value.

Optionally, the classifier is generated according to a set of rules that are manually entered by the user (e.g., the expert physician performing multiple procedures on multiple patients). The set of rules may be automatically learned by code that analyses the behavior of the physician. The set of rules may depend on features other than those measured during the procedure, for example, the patient profile and/or settings of the treatment device according to the type of hardware of the treatment device. For example, the set of rules may define how intervention target regions are selected according to whether the patient is male or female, according to the age of the patient, according to the weight of the patient, according to ethnic origin of the patient, and according to the make and model of the treatment device. The gender and/or other personal data of the patient and/or treatment device type may be manually entered by a user (e.g., via the GUI) and/or automatically extracted by code from the electronic medical record of the patient. At 162, the intervention procedure is in progress. At least one tissue region of the organ is treated, for example, ablated.

Additional electrical readings and/or transformations thereof are obtained by respective electrodes 214 located within the portion of the organ. The additional electrical reading(s) are obtained after tissue region(s) of the organ are treated (e.g. ablated).

The additional electrical reading(s) are mapped to the 3D image of the portion of the organ

Optionally, updated and/or additional extracted features of other data is received, for example, features indicative of changes based on the intervention.

At 164, an updated association of the additional electrical reading(s) and/or transformations thereof, and a certain cluster is defined with an updated label indicative of the adjusted treatment of the intervention target regions (optionally type of the cluster). For example, the user manually designates the additional electrical readings(s) into one of the existing intervention target region types with the GUI.

It is noted that previously collected electrical reading(s) may be re-classified (e.g., by the user via the GUI) from the original treatment of intervention target type into a new treatment of intervention target type, or from the original treatment of intervention target to a non-intervention target, or from the original non-intervention target into treatment of one of the intervention target types.

At 166, data is received for training the classifier to perform prediction and/or estimation, as described with reference to act 122 of FIG. 1A. The data may be manually entered by user and/or automatically computed based on data received via an interface (e.g., software interface). For example, the ablation time may be obtained from an interface of an ablation device, and/or the total procedure time may be computed according to a clock and indications of the start and end of the procedure.

At 168, the classifier(s) is updated according to the additional electrical readings and/or transformation thereof and updated label indicative of the adjusted treatment of the intervention region(s), and optionally in view of the additional and/or updated extracted features (e.g., indicative of changes occurring due to the procedure).

It is noted that the training of the classifier described with reference to act 102 of FIG. 1A may occur after the invention procedure is performed, for example, after act 124.

Moreover, it is noted that the data collected during the intervention procedure described with reference to FIG. 1A may be used to update the classifier based on the process described with reference to FIG. 1B. New electrical readings and/or transformation thereof and an indication of treatment of a region identified as the intervention target region (wherein the intervention target region is associated with a subset of the electrical readings) may be provided as input for updating the trained classifier. The trained classifier may be repeatedly updated with data from additional intervention procedures, to increase the size of the training data, which increases the accuracy of the classifier.

Optionally, medical data is obtained for the sample individuals at least a time interval after completion of the intervention procedure during which the electrical readings and/or transformation thereof were collected, for example, at least 1 month, 6 months, 1 year, or 2 years, or other values. The medical data may be manually entered by a user (e.g., via the GUI) and/or automatically extracted by code from the patient medical record. Sample individuals associated with an indication of an unsuccessful procedure outcome may be removed from the trained classifier. The trained classifier is updated to exclude the data of the sample individuals associated with the indication of unsuccessful treatment. Alternatively, the classifier is updated with an indication that the data (i.e., electrical readings and/or transformation thereof, and/or indication(s) of intervention target region(s)) is associated with an unsuccessful outcome. The updated classifier is less likely to select intervention target regions associated with unsuccessful outcomes. Alternatively, the classifier is updated with an indication that the data (i.e., electrical readings and/or transformation thereof, and/or indication(s) of intervention target region(s)) is associated with a successful outcome, or a value indicative of a degree of success or failure (e.g., on a scale of 1 to 10, where 10 is very successful and 1 is a complete failure). The classifier selects instructions for treatment of intervention target regions based on likelihood of success, as described herein.

At 170, the features described with reference to acts 162-168 are iterated, as new electrical readings are obtained during the intervention procedure.

Reference is now made to FIG. 3, which is a schematic of a 3D image 302 of a left atrium 304 and pulmonary veins 306A-D and regions 308A-D from which electrical readings for identification of instructions for treatment of an intervention target region were obtained by respective electrodes, in accordance with some embodiments of the present invention. The schematic of FIG. 3 is provided as an example, to illustrate the process of identifying by the classifier(s) instructions for treatment of one or more regions in a portion of an organ identified as an intervention target, for example, ablation of an ablation line.

3D image 302 is computed based on MRI imaging data collected from an MRI machine that imaged at least the left atrium and connecting pulmonary vessels of the target individual. Is it noted that other 3D image representations may be implemented, for example, based on reconstructing the 3D image from electrical readings indicative of the location of electrodes within the organ, for example, performed by navigation system 236 based on outputs of pad-electrodes 228, as described herein.

Electrical readings 310A-D are obtained from corresponding locations 308A-D of the left inferior pulmonary vein. Each electrical readings 310A-D represents a different signal obtained from a different location (308A-D) within the heart, for example, by pulling back a catheter with electrodes that measure the electrical readings.

Pulmonary vein signals of electrical readings 310A-D are each denoted by a respective dashed line 312A-D.

Electrical readings 310A-D include atrial signals 314A-D.

Set of signals denoted by arrow 316 are collected by lead I.

Set of signals denoted by arrow 318 are collected by an electrode(s) located within the coronary sinus (CS).

Electrical reading 310D is obtained from location 308D. Electrical reading 310D includes pulmonary signal 312D, and atrial signal 314D. It is noted that atrial signal 314D is significantly smaller than pulmonary signal 312D. A wide low amplitude atrial signal 314D is followed by a sharp pulmonary vein signal 312D. Electrical reading 310D is labeled (e.g., manually tagged) as being located within the pulmonary vein according to multiple (e.g., all) labeling criteria.

Electrical reading 310C is obtained from location 308C. Electrical reading 310C includes pulmonary signal 312C, and atrial signal 314C. It is noted that atrial signal 314C and pulmonary signal 312C are approximately the same size, and both signals are significantly larger than the atrial and pulmonary signals of electrical reading 310D. Electrical reading 310C is labeled as located on the border between the vein and atrium according to the criteria of the atrial and pulmonary signal being approximately of equal sizes.

Electrical reading 310B is obtained from location 308B. Electrical reading 310B includes pulmonary signal 312B, and atrial signal 314B. It is noted that atrial signal 314B and pulmonary signal 312B are both of a maximum size in comparison to other electrical readings obtained from other locations. A sharp farfield atrial signal 314B precedes the sharp pulmonary vein signal 312B without an isoelectric line in between. According to other criteria (different than that used to label the other electrical readings as located on the border between the vein and atrium) electrical reading 310B is labeled as located on the border between the vein and atrium according to maximal size.

Electrical reading 310A is obtained from location 308A. Electrical reading 310A includes atrial signal 314A. It is noted that no pulmonary signal is depicted. According to other criteria (different than that used to label the other electrical readings as located on the border between the vein and atrium) electrical reading 310A is labeled as located on the border between the vein and atrium according the location where the pulmonary signal disappears.

It is noted that multiple sets of electrical readings may be obtained around the circumference of the inner wall of the lumen (e.g., pulmonary vein, atrium near pulmonary vein), for example, by repeating the pullback of the catheter at different points around the circumference (e.g., 3-6 times, or other numbers).

Respective ablation rings may be defined according to the location of the electrical readings, based on one or more of the set of criteria that define the border between the pulmonary vein and the left atrium.

The electrical readings may be clustered according to one or more sets of criteria, described with reference to electrical readings 310A-C, for example, by the user manually selecting each electrical reading and assigning the respective electrical reading to one of the clusters according to respective criteria, and/or by the user manually drawing a boundary delineating the intervention target region relative to electrical readings 310A-C on a GUI presenting the 3D image and regions of electrical readings 310A-C.

The classifier(s) described herein may be trained according to associations between the electrical readings and/or transformations of the electrical readings and regions in the heart identified as ablation target regions (e.g., the associated clustering labels indicative of each type of ablation target region). Alternatively or additionally, ablation lines (i.e., intervention target regions) are identified by the trained classifier(s) according to newly received electrical readings obtained by electrodes located within the heart of a new target patient.

The user may indicate a preference as to which criteria to use for identification of the ablation ring (e.g., manually select from a list of available criteria, via the GUI). Alternatively, multiple identified ablation rings are presented on the 3D image, where each ablation ring is identified according to respective criteria. The user may select one of the multiple presented ablation rings (e.g., click on the ring via the GUI). The non-selected rings may be removed from the 3D image.

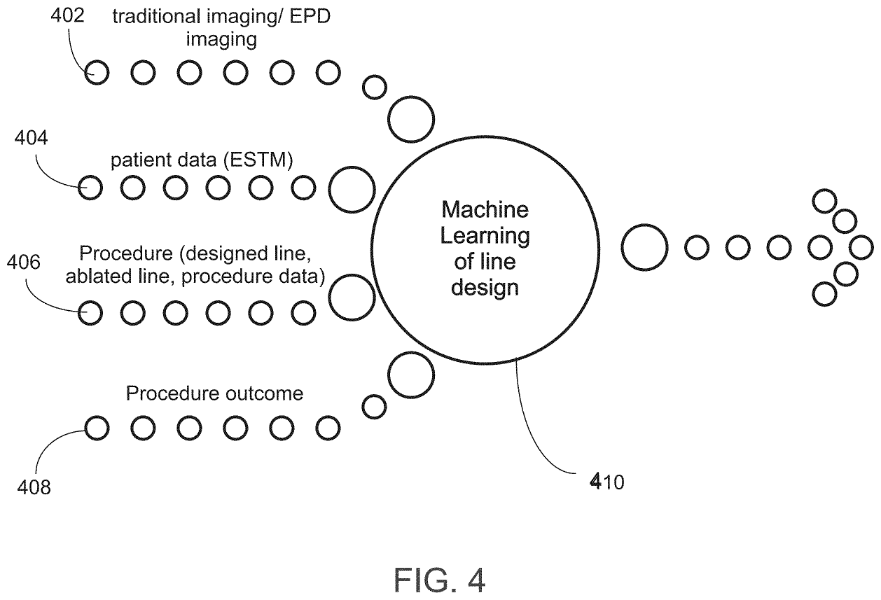

Reference is now made to FIG. 4, which is a schematic depicting an exemplary process of generating a classifier(s) for identifying instructions for treatment of region(s) in a portion of an organ identified as intervention target region(s), in accordance with some embodiments of the present invention. The generation of the classifier described with reference to FIG. 4 may be implemented by one or more components of system 200 described with reference to FIG. 2 (e.g., hardware processors(s) 204 of computing device 202 executing code stored in data storage device 206). The generation of the classifier may be based on the feature described with reference to FIG. 1B. The following components are received:
Image(s) 402, for each of multiple sample patients, optionally 3D images, of the portion of the organ of the target patient corresponding to regions of the organ from which electrical readings are obtained by the electrodes of the catheter. The 3D images may include pre-acquired 3D anatomical images that are mapped to the locations of the electrodes, and/or the 3D images may be computed based on navigation data obtained by electrodes within the heart and/or by pad-electrodes, as described herein.
Additional features for each of the multiple sample patients, for example, as described with reference to act 106 of FIG. 1A and/or act 154 of FIG. 1B.
Procedure data 406 obtained for each of multiple sample patients. The procedure data 406 includes defined associations between analyzed electrical readings and/or transformations thereof and treatment of regions in the portion of the organ identified as interventional target regions. The defined association may be manually designated by the user performing each procedure, for example, within a GUI presenting the 3D image of the portion of the organ, which may include the regions from which electrical readings were obtained, the user manually delineates the manually identified intervention target on the 3D image.

Additional procedure data includes the electrical readings and/or transformations thereof, obtained before and/or during the intervention treatment procedure, adjustment of the designated intervention target region, procedure time, ablation energy, ablation time, and/or other procedure related data, for example, as described with reference to acts 162-166 of FIG. 1B.
Procedure outcome 408 indicative of the success (or lack of success) of the procedure for each of the multiple sample individuals, for example, as described herein.

The classifier(s) is generated 410 to identify for a new target patient, instructions for treatment of one or more regions in a portion of an organ as intervention target region(s) based on the input data 402-408.

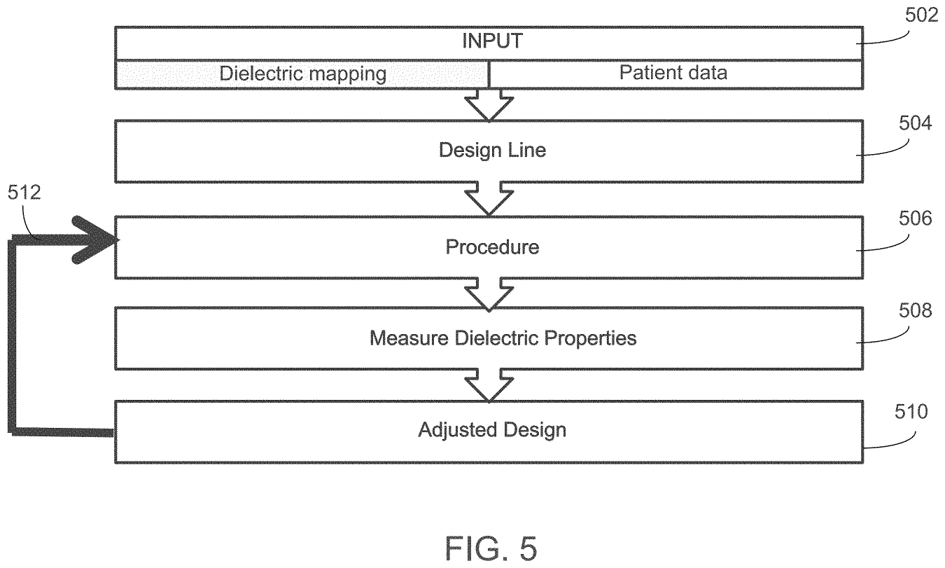

Reference is now made to FIG. 5, which is a flowchart depicting an exemplary process of identifying instructions for treatment of a region in a portion of an organ identified as an ablation line target region, in accordance with some embodiments of the present invention. The process described with reference to FIG. 5 may be implemented by one or more components of system 200 described with reference to FIG. 2 (e.g., hardware processors(s) 204 of computing device 202 executing code stored in data storage device 206), and/or based on one or more features of the processes described with reference to FIGS. 1A-1B.

At 502, pre-procedure data is received. The pre-procedure data includes electrical readings obtained by electrodes located within the portion of the organ, for example, as described with reference to act 104 of FIG. 1A. The pre-procedure data may include additional features, for example, as described with reference to act 106 of FIG. 1A.

At 504, the classifier(s) identify instructions for treatment of one or more regions in the portion of the organ identified as the ablation line target region, for example, as described with reference to act 108 of FIG. 1A.

The classifier is based on previously associated electrical readings and/or transformation of the electrical readings (and optionally one or more features) and treatment of regions in the portion of the organ identified as intervention target regions.

At 506, the ablation procedure is in progress. One or more tissue regions are ablated according to the identified ablation line target region.

At 508, additional electrical readings and/or transformations thereof and/or features are received, for example, as described with reference to acts 114-116 of FIG. 1A.

At 510, adjusted instructions for treatment of the ablation line target region are identified based on the additional electrical readings and/or transformations thereof and/or features, for example, as described with reference to act 118 FIG. 1A.

At 512, acts 506-510 are iterated, to dynamically adjust the instructions for treatment of the ablation line target region during the ablation procedure based on additional electrical readings and/or transformations thereof and/or features, for example, as described with reference to act 124 of FIG. 1A.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

It is expected that during the life of a patent maturing from this application many relevant electrical readings will be developed and the scope of the term electrical reading is intended to include all such new technologies a priori.

As used herein the term “about” refers to ±10%.

The terms “comprises”, “comprising”, “includes”, “including”, “having” and their conjugates mean “including but not limited to”. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A computer implemented method of providing a client terminal with instructions for treatment of at least a portion of an organ of a patient, the method comprising:
receiving electrical readings obtained by electrodes located within the portion of the organ;
identifying by at least one classifier of a region in the portion of the organ as an intervention target region;
identifying, using the at least one classifier, instructions for treatment of the region identified as an intervention target region based on electrical readings or a transformation thereof previously associated with treatment of intervention target regions in the portion of the organ of other patients; and
marking on an image of the portion of the organ presented on a display, the instruction for treatment of the region identified by the classifier as an intervention target region.

2-54. (canceled)

55. The method of claim 1, wherein the instructions are provided as one or more members selected from the group consisting of: a text message presented on the display indicating how to treat the intervention target region, an animation simulating treatment of the intervention target region, a video captured of another user previously performing a treatment, and an audio recording of instructions how to treat the intervention target region.

56. The method of claim 1, wherein the classifier identifies the instructions according to a hardware type of a treatment device for performing the treatment.

57. The method of claim 56, wherein the instructions for treatment comprise one or more treatment parameters for application to the identified intervention target region, wherein the one or more treatment parameters are selected for a treatment modality selected from the group consisting of: probe pressure, heating, cooling, cardiac pacing, defibrillation, radiofrequency energy application, radiofrequency ablation, cryo application, cryo ablation, other energy delivery, and combinations of the aforementioned.

58. The method of claim 1, further comprising reconstructing the image of the portion of the organ based on said previously associated electrical readings or the transformation thereof.

59. The method of claim 1, wherein the received electrical readings are of a first type, the image is computed based on a second type of electrical reading, and the electrical readings of the first type are associated with locations at which the received electrical readings were obtained by the electrodes, the locations associated with the electrical readings of the first type are mapped to corresponding locations of the image.

60. The method of claim 59, wherein the first type is an electrogram and the second type is an impedance reading.

61. The method of claim 1, wherein the classifier identifies instructions for treatment of the region based on impedance electrical readings previously associated with treatment of intervention target regions in the portion of the organ of other patients.

62. The method of claim 1, wherein the received electrical readings comprise position electrical readings indicative of anatomical position of at least one of the electrodes when the position electrical readings are read, wherein each anatomical position is associated with an anatomical structure.

63. The method of claim 1, wherein the classifier identifies the region as an intervention target region based on combinations of anatomical positions and impedance electrical readings, said combinations being previously associated with treatment of intervention target regions in the portion of the organ of other patients.

64. The method of claim 1, wherein the classifier identifies instructions for treatment of the region based on cardiac-phased electrical readings, previously associated with treatment of intervention target regions in the portion of the organ of other patients.

65. The method of claim 1, wherein the classifier identifies the instructions based on respiratory-phased electrical readings, previously associated with treatment of intervention target regions in the portion of the organ of other patients.

66. The method of claim 1, wherein the classifier identifies instructions for treatment of the region based on electrogrammed electrical readings, previously associated with treatment of intervention target regions in the portion of the organ of other patients.

67. The method of claim 1, wherein each of the received electrical readings is associated with a time stamp indicative of the time at which the respective electrical reading was obtained by the electrodes, wherein the electrical readings having time stamps within defined time windows indicative of an approximate simultaneous time of acquisition are clustered to time window clusters, wherein the classifier identifies the instructions for treatment of intervention target region according to time window clusters of electrical readings or a transformation thereof previously associated with treatment of intervention target regions in the portion of the organ of other patients.

68. The method of claim 1, wherein the classifier identifies the region as an intervention target region based on previously observed associations between scores indicative of success of the treatment of intervention target regions in the portion of the organ of other patients, and wherein the identifying instructions for treatment by the classifier is performed according to maximization of a predicted likelihood of success of a treatment performed according to the identified intervention target region.

69. The method of claim 1, wherein the instructions for treatment of the target intervention region denotes instructions for ablation treatment of an ablation region, and wherein the ablation region is marked on the image of the portion of the organ.

70. The method of claim 1, further comprising identifying, by at least one classifier, a second region in the portion of the organ identified as a non-intervention region, in which intervention is prohibited, wherein the classifier is based on observed associations between previously analyzed electrical readings and regions in the portion of the organ previously identified as non-intervention regions.

71. The method of claim 70, further comprising identifying, by at least one classifier, instructions for avoidance of treatment of the second region.

72. The method of claim 1, further comprising:
receiving additional electrical readings obtained by the electrodes after at least one tissue region of the organ is treated;
re-identifying by the at least one classifier updated instructions for treatment of a region in the portion of the organ identified as an updated intervention target region, wherein the instructions for treatment of region are updated by the at least one classifier based on observed associations between previously analyzed electrical readings and treatment of regions in the portion of the organ previously identified as updated intervention target regions; and
marking on the image of the portion of the organ, instructions for treatment of an updated region identified by the at least one classifier as an updated target region.

73. The method of claim 1, further comprising:
receiving an indication of a treatment region in which an intervention treatment was performed within the portion of the organ;
computing an adjustment to the instructions for treatment of the intervention target region identified by the at least one classifier according to the treatment region and the received indication; and
marking the adjusted instructions on the image.

74. The method according to claim 72, further comprising dynamically computing an indication of a recommendation for proceeding in the treatment to treat the intervention target region according to the updated intervention target region, for presentation in association with the image.

75. The method according to claim 72, wherein the additional electrical readings are obtained after at least a portion of an interventional procedure is performed according to the location of the region identified by the classifier as the intervention target region.

76. The method of claim 1, wherein the electrical readings are selected from the group consisting of readings of: voltage, impedance, endocardial electrical activity, electrical activity, dielectric property, S-parameter and combinations of the aforementioned.

77. The method of claim 1, further comprising generating electrical fields affecting the electrical readings, wherein the electrical fields are generated by one or more of the following: patch electrodes located externally to the body of the target individual that apply a plurality of alternating currents each at a respective frequency, electrodes on an intra-body catheter located in proximity to the electrodes that obtain the electrical readings, and electrodes on an intra-body catheter located in a predefined anatomical region.

78. The method of claim 1, further comprising:
receiving data indicative to at least one tissue property obtained by at least one sensor located within the portion of the organ;
wherein identifying instructions for treatment of the region by the at least one classifier is further based on observed associations between the at least one tissue property indicated by the received data and treatment of regions in the portion of the organ of other patients.

79. The method according to claim 78, wherein the at least one tissue property is selected from the group consisting of: molecular structure, IR reflectance, NIR reflectance, Ho Yag reflectance, pH, Ion concentration, Reactance, tissue thickness, scars and combinations of the aforementioned.

80. The method of claim 1, further comprising:
receiving manually entered instructions for treatment of a designated intervention target region;
correlating the instructions for treatment of the intervention target region identified by the at least one classifier with the manually entered instructions for treatment of the intervention target region according to a correlation requirement; and
computing an adjustment to the manually entered instructions for treatment of the intervention target region that satisfies the correlation requirement; and marking the adjusted instruction on the image.

81. The method of claim 1, wherein the instructions for treatment include:
power of ablation and time of application of ablation energy for performing an intervention procedure identified based on observed associations between intervention target regions and previously analyzed powers of ablation and times of application of ablation energy.

82. A system for providing a client terminal with instructions for treatment of at least a portion of an organ of a patient, the system comprising:
a non-transitory memory having stored thereon a code for execution by at least one hardware processor, the code comprising:
code for receiving electrical readings obtained by electrodes located within the portion of the organ,
code for identifying by at least one classifier a region in the portion of the organ as an intervention target region,
code for identifying, using the at least one classifier, instructions for treatment of the region identified as an intervention target region based on electrical readings or a transformation thereof previously associated with treatment of intervention target regions in the portion of the organ of other patients; and
code for marking on an image of the portion of the organ presented on a display, the instructions for treatment of the location of a region identified by the classifier as an intervention target region.

83. A computer program product comprising a non-transitory computer readable storage medium storing program code thereon for implementation by a processor of a computing device for providing a client terminal with instructions for treatment of at least a portion of an organ of a patient, the program code comprising:
instructions to receive electrical readings obtained by electrodes located within the portion of the organ,
instructions to identify by at least one classifier instructions for treatment of a region in the portion of the organ
instructions to identify, using the at least one classifier, instructions for treatment of the region identified as an intervention target region based on electrical readings or a transformation thereof previously associated with treatment of intervention target regions in the portion of the organ of other patients; and
instructions to mark on an image of the portion of the organ presented on a display, the instructions for treatment of the location of a region identified by the classifier as an intervention target region.
